(19) United States
(12) Patent Application Publication
(10) Pub. No.: US 2020/0199638 A1
(43) Pub. Date: Jun. 25, 2020

(54) Starch Extraction Method

(71) Applicant: Novozymes A/S, Bagsvaerd (DK)

(72) Inventors: Thomas Patrick Gibbons, Wake Forest, NC (US); Oscar Pastor Ferrer, Benifaio (SP); Bernardo Vidal, Wake Forest, NC (US)

(73) Assignee: Novozymes A/S, Bagsvaerd (DK)

(21) Appl. No.: 16/612,449

(22) PCT Filed: May 28, 2018
(86) PCT No.: PCT/EP2018/063907

§ 371 Date: Nov. 11, 2019

(30) Foreign Application Priority Data
May 30, 2017 (EP) 17382311.3

(51) Int. Cl. C12P 19/14

(52) U.S. Cl. CPC C12P 19/14

(57) ABSTRACT

The present invention provides improved methods for enzyme-assisted corn wet milling. Also provided are corn kernel wet milling systems which may be used to perform the methods of the invention.

### FIELD OF THE INVENTION

The present invention relates to improved methods for using hydrolytic enzymes to increase the yield of starch and gluten in corn wet milling processes. Furthermore, the present invention relates to corn wet milling systems useful for practising the methods of the invention and to fine fiber fractions provided using the methods.

### BACKGROUND OF THE INVENTION

Conventional wet milling of corn is a process designed for the recovery and purification of starch and several coproducts including germ, gluten and fiber.

Fiber is the least valuable coproduct, so the industry has put substantial effort into increasing the yield of the more valuable products, such as starch and gluten, while decreasing the fiber fraction. High quality starch is valuable as it can be used for a variety of commercial purposes after further processing to products such as dried starch, modified starch, dextrins, sweeteners and alcohol. Gluten is usually used for animal feed, as corn gluten meal (Around 60% protein) or corn gluten feed (Around 20% protein).

The wet milling process can vary significantly dependent on the specific mill equipment used, but usually the process include: grain cleaning, steeping, grinding, germ separation, a second grinding, fiber separation, gluten separation and starch separation. After cleaning the corn kernels, they are typically softened by soaking in water or in a dilute SO₂ solution under controlled conditions of time and temperature. Then, the kernels are grinded to break down the pericarp and the germ is separated from the rest of the kernel. The remaining slurry, mainly consisting of fiber, starch and gluten is finely ground and screened in a fiber washing process, to separate the fiber from starch and gluten, before the gluten and starch is separated and the starch can be purified in a washing/filtration process.

The use of enzymes in several steps of the wet milling process has been suggested, such as the use of enzymes for the steeping step of wet milling processes. The commercial enzyme product Steepzyme® (available from Novozymes NS) has been shown suitable for the first step in wet milling processes, i.e., the steeping step where corn kernels are soaked in water.

More recently, “enzymatic milling”, a modified wet milling process that uses proteases to significantly reduce the total processing time during corn wet milling and eliminates the need for sulfur dioxide as a processing agent, has been developed. Johnston et al., Cereal Chem, 81, p. 626-632 (2004).

U.S. Pat. No. 6,566,125 discloses a method for obtaining starch from maize involving soaking maize kernels in water to produce soaked maize kernels, grinding the soaked maize kernels to produce a ground maize slurry, and incubating the ground maize slurry with enzyme (e.g., protease).

U.S. Pat. No. 5,066,218 discloses a method of milling grain, especially corn, comprising cleaning the grain, steeping the grain in water to soften it, and then milling the grain with a cellulase enzyme.

WO 2002/000731 discloses a process of treating crop kernels, comprising soaking the kernels in water for 1-12 hours, wet milling the soaked kernels and treating the kernels with one or more enzymes including an acidic protease.

WO 2002/000911 discloses a process of starch gluten separation, comprising subjecting mill starch to an acidic protease.

WO 2002/002644 discloses a process of washing a starch slurry obtained from the starch gluten separation step of a milling process, comprising washing the starch slurry with an aqueous solution comprising an effective amount of acidic protease.

WO 2014/082566 and WO 2014/082564 disclose cellulolytic compositions for use in wet milling.

While the art has investigated the effect of using enzymes in corn wet milling, during steeping/soaking of corn kernels, during grinding of the corn kernels and in starch gluten separation, there is still a need for improved enzyme technology that may improve the recovery of starch and gluten in corn wet milling.

### SUMMARY OF THE INVENTION

The present invention relates to a method for improving starch yield and/or gluten yield from corn kernels in a wet milling process, comprising
a) separating starch and/or gluten from fiber to provide a fiber fraction,
b) providing a fine fiber fraction by separating coarse fibers of the fiber fraction from fine fibers of the fiber fraction; and
c) contacting the fine fiber fraction with one or more hydrolytic enzymes.

In a second aspect, the invention relates to a corn kernel wet milling system comprising
i) a fiber washing system (F)
ii) a fiber press (P),
iii) means for dosing one or more hydrolytic enzymes; and
iv) a space (V₁);

wherein said fiber washing system (F), said fiber press (P) and said space (V₁) are interconnected to allow a flow of a fiber fraction from the fiber washing system to the press, a flow of fiber press filtrate or a fine fiber fraction from the press to the space (V₁);

wherein the said means for dosing one or more hydrolytic enzymes is configured for dosing said enzymes to the fiber press filtrate or the fine fiber fraction; and

wherein said space (V₁) has a volume of at least 100 m³/1000 tonne corn processed per day.

In a third aspect, the invention relates to a fiber fraction, such as a fine fiber fraction, which is obtainable or is obtained by the method according to the invention.

### BRIEF DESCRIPTION OF THE FIGURES

The present invention and in particular preferred embodiments according to the invention will be described in more detail with reference to the accompanying figures. The figures show ways of implementing the present invention and are not to be construed as being limiting to other possible embodiments falling within the scope of the attached claim set.

FIG. 1 schematically illustrates an embodiment of a corn wet milling process according to the present invention.

FIG. 2 schematically illustrates an embodiment of a counter current fiber wash system used in the process of the present invention.

FIG. 3 schematically illustrates a fiber washing procedure in which a space (V) is arranged between the screening units of the system.

FIG. 4 shows average % Insoluble Solids Recovered Based on Starting Material

FIG. 5 shows average % Starch in Residual Fine Fiber.

FIG. 6 shows average % Protein in Residual Fine Fiber.

### DETAILED DESCRIPTION

It is an object of the present invention to provide a fiber washing system optimized for the use of hydrolytic enzymes. Furthermore, it is an object of the present invention to provide a method that improves starch and gluten yield from corn kernels in a wet milling process. Other benefits of the invention include improved fiber dewatering and an anti-foaming effect.

The Wet Milling Process:

Corn kernels are wet milled in order to open up the kernels and separate the kernels into its four main constituents: starch, germ, fiber and gluten.

The wet milling process can vary significantly from mill to mill, however conventional wet milling usually comprises the following steps:

1. Steeping and germ separation,

2. Fiber washing, pressing and drying,

3. Starch/gluten separation, and

4. Starch washing.

1. Steeping, Grinding and Germ Separation

Corn kernels are softened by soaking in water for between about 30 minutes to about 48 hours, preferably 30 minutes to about 15 hours, such as about 1 hour to about 6 hours at a temperature of about 50° C., such as between about 45° C. to 60° C. During steeping, the kernels absorb water, increasing their moisture levels from 15 percent to 45 percent and more than doubling in size. The optional addition of e.g. 0.1 percent sulfur dioxide (SO₂) and/or NaHSO₃ to the water prevents excessive bacteria growth in the warm environment. As the corn swells and softens, the mild acidity of the steepwater begins to loosen the gluten bonds within the corn and release the starch. After the corn kernels are steeped they are cracked open to release the germ. The germ contains corn oil. The germ is separated from the heavier density mixture of starch, gluten and fiber essentially by “floating” the germ segment free of the other substances under closely controlled conditions. This method serves to eliminate any adverse effect of traces of corn oil in later processing steps.

2. Fiber Washing, Pressing and Drying

To get maximum starch and gluten recovery, while keeping any fiber in the final product to an absolute minimum, it is necessary to wash the free starch and gluten from the fiber during processing. The free starch and gluten is separated from fiber during screening (washing) and collected as mill starch. The remaining fiber is then pressed to decrease the water content and dried.

3. Starch Gluten Separation

The starch-gluten suspension from the fiber-washing step, called mill starch, is separated into starch and gluten. Gluten has a low density compared to starch. By passing mill starch through a centrifuge, the gluten is readily spun out.

4. Starch Washing

The starch slurry from the starch separation step contains some insoluble protein and much of solubles. They have to be removed before a top quality starch (high purity starch) can be made. The starch, with just one or two percent protein remaining, is diluted, washed 8 to 14 times, re-diluted and washed again in hydroclones to remove the last trace of protein and produce high quality starch, typically more than 99.5% pure.

Products of Wet Milling:

Wet milling can be used to produce, without limitation, corn steep liquor, corn gluten feed, germ, corn oil, corn gluten meal, corn starch, modified corn starch, syrups such as corn syrup, and corn ethanol.

### Definition of Enzymes

Arabinofuranosidases/polypeptide with arabinofuranosidase activity: The term “arabinofuranosidase” means an alpha L-arabinofuranoside arabinofuranohydrolase (EC 3.2.1.55) that catalyzes the hydrolysis of terminal non-reducing alpha-L-arabinofuranoside residues in alpha-L-arabinosides. The enzyme acts on alpha-L-arabinofuranosides, alpha-L-arabinans containing (1,3)- and/or (1,2)- and/or (1,5)-linkages, arabinoxylans, and arabinogalactans. Alpha-L arabinofuranosidase is also known as arabinosidase, alpha-arabinosidase, alpha-L-arabinosidase, alphaarabinofuranosidase, polysaccharide alpha-L-arabinofuranosidase, alpha-L-arabinofuranoside hydrolase, L-arabinosidase, or alpha-L-arabinanase. Arabinofuranosidase activity can be determined using 5 mg of medium viscosity wheat arabinoxylan (Megazyme International Ireland, Ltd., Bray, Co. Wicklow, Ireland) per ml of 100 mM sodium acetate pH 5 in a total volume of 200 μl for 30 minutes at 40° C. followed by arabinose analysis by AMINEX® HPX-87H column chromatography (Bio-Rad Laboratories, Inc., Hercules, Calif., USA).

Beta-glucosidase/polypeptide with beta-glucosidase activity: The term “beta-glucosidase” means a beta-D-glucoside glucohydrolase (E.C. 3.2.1.21) that catalyzes the hydrolysis of terminal non-reducing beta-D-glucose residues with the release of beta-D-glucose. Beta-glucosidase activity can be determined using pnitrophenyl-beta-D-glucopyranoside as substrate according to the procedure of Venturi et al., 2002, J. Basic Microbiol. 42: 55-66. One unit of beta-glucosidase is defined as 1.0 μmole of pnitrophenolate anion produced per minute at 25° C., pH 4.8 from 1 mM p-nitrophenyl-beta-Dglucopyranoside as substrate in 50 mM sodium citrate containing 0.01% TWEEN® 20.

Beta-xylosidase/polypeptide with beta-xylosidase activity: The term “beta-xylosidase” means a beta-D-xyloside xylohydrolase (E.C. 3.2.1.37) that catalyzes the exo-hydrolysis of short beta (1→4)-xylooligosaccharides to remove successive D-xylose residues from non-reducing termini. Beta-xylosidase activity can be determined using 1 mM p-nitrophenyl-beta-D-xyloside as substrate in 100 mM sodium citrate containing 0.01% TWEEN® 20 at pH 5, 40° C. One unit of beta-xylosidase is defined as 1.0 μmole of p15 nitrophenolate anion produced per minute at 40° C., pH 5 from 1 mM p-nitrophenyl-beta-Dxylosidein 100 mM sodium citrate containing 0.01% TWEEN® 20.

Cellobiohydrolase/polypeptide with cellobiohydrolase activity: The term “cellobiohydrolase” means a 1,4-beta-D-glucan cellobiohydrolase (E.C. 3.2.1.91 and E.C. 3.2.1.176) that catalyzes the hydrolysis of 1,4-beta-D15 glucosidic linkages in cellulose, cellooligosaccharides, or any beta-1,4-linked glucose containing polymer, releasing cellobiose from the reducing end (cellobiohydrolase I) or non-reducing end (cellobiohydrolase II) of the chain (Teen, 1997, Trends in Biotechnology 15: 160-167; Teeri et al., 1998, Biochem. Soc. Trans. 26: 173-178). Cellobiohydrolase activity can be determined according to the procedures described by Lever et al., 1972, Anal. Biochem. 47: 273-279; van Tilbeurgh et al., 1982, FEBS Letters 149: 152-156; van Tilbeurgh and Claeyssens, 1985, FEBS Letters 187: 283-288; and Tomme et al., 1988, Eur. J. Biochem. 170: 575-581.

Cellulolytic enzyme or cellulase/polypeptide with cellulase activity or cellulolytic activity: The term “cellulolytic enzyme” or “cellulase” means one or more (e.g., several) enzymes that hydrolyze a cellulosic material, which comprise any material comprising cellulose, such as fiber. Cellulytic enzymes include endoglucanase(s) (E.C. 3.2.1.4), cellobiohydrolase(s) (E.C. 3.2.1.91 and E.C. 3.2.1.150), beta-glucosidase(s) (E.C. 3.2.1.21), or combinations thereof. The two basic approaches for measuring cellulolytic enzyme activity include: (1) measuring the total cellulolytic enzyme activity, and (2) measuring the individual cellulolytic enzyme activities (endoglucanases, cellobiohydrolases, and beta-glucosidases) as reviewed in Zhang et al., 2006, Biotechnology Advances 24: 452-481. Total cellulolytic enzyme activity can be measured using insoluble substrates, including Whatman No 1 filter paper, microcrystalline cellulose, bacterial cellulose, algal cellulose, cotton, pretreated lignocellulose, etc. The most common total cellulolytic activity assay is the filter paper assay using Whatman No 1 filter paper as the substrate. The assay was established by the International Union of Pure and Applied Chemistry (IUPAC) (Ghose, 1987, Pure Appl. Chem. 59: 257-68).

Cellulolytic enzyme activity can be determined by measuring the increase in production/release of sugars during hydrolysis of a cellulosic material by cellulolytic enzyme(s) under the following conditions: 1-50 mg of cellulolytic enzyme protein/g of cellulose in pretreated corn stover (PCS) (or other pretreated cellulosic material) for 3-7 days at a suitable temperature such as 40° C.-80° C., e.g., 40° C., 45° C., 50° C., 55° C., 60° C., 65° C., 70° C., 75° C., or 80° C., and a suitable pH, such as 4-9, e.g., 4.0, 4.5, 5.0, 5.5, 6.0, 6.5, 7.0, 7.5, 8.0, 8.5, or 9.0, compared to a control hydrolysis without addition of cellulolytic enzyme protein. Typical conditions are 1 ml reactions, washed or unwashed PCS, 5% insoluble solids (dry weight), 50 mM sodium acetate 5 pH 5, 1 mM MnSO4, 50° C., 55° C., or 60° C., 72 hours, sugar analysis by AMINEX® HPX-87H column chromatography (Bio-Rad Laboratories, Inc., Hercules, Calif., USA).

Hydrolytic enzymes or hydrolase/polypeptide with hydrolase activity: “Hydrolytic enzymes” refers to any catalytic protein that use water to break down substrates. Hydrolytic enzymes include cellulases (EC 3.2.1.4), xylanases (EC 3.2.1.8) arabinofuranosidases (EC 3.2.1.55 (Non-reducing end alpha-L-arabinofuranosidases); EC 3.2.1.185 (Non-reducing end beta-L-arabinofuranosidases) cellobiohydrolase I (EC 3.2.1.150), cellobiohydrolase II (E.C. 3.2.1.91), cellobiosidase (E.C. 3.2.1.176), beta-glucosidase (E.C. 3.2.1.21), beta-xylosidases (EC 3.2.1.37).

Xylanases/polypeptide with xylanase activity: The term “xylanase” means a 1,4-beta-D-xylan-xylohydrolase (E.C. 3.2.1.8) that catalyzes the endohydrolysis of 1,4-beta-D-xylosidic linkages in xylans. Xylanase activity can be determined with 0.2% AZCL-arabinoxylan as substrate in 0.01% TRITON® X-100 and 200 mM sodium phosphate pH 6 at 37° C. One unit of xylanase activity is defined as 1.0 μmole of azurine produced per minute at 37° C., pH 6 from 0.2% AZCL-arabinoxylan as substrate in 200 mM sodium phosphate pH 6.

### Other Definitions

In the present context, terms are used in manner being ordinary to a skilled person. Some of these terms are elucidated below:

Corn kernel: A variety of corn kernels are known, including, e.g., dent corn, flint corn, pod corn, striped maize, sweet corn, waxy corn and the like.

Some corn kernels has an outer covering referred to as the “Pericarp” that protects the germ in the kernels. It resists water and water vapour and is undesirable to insects and microorganisms. The only area of the kernels not covered by the “Pericarp” is the “Tip Cap”, which is the attachment point of the kernel to the cob.

Corn kernel mass: is preferably used to reference a mass comprising fiber, gluten and starch, preferably achieved by steaming and grinding crop kernels and separating a mass comprising fiber, gluten and starch from germs. As the corn kernel mass move through the fiber washing, it is separated into several fractions, including first (s) and second fractions (f). Hence, “fractions of corn kernel mass” and “one or more fractions of corn kernel mass” refer to these first (s) and second fractions (f).

Expression: The terms “expression” and “expressed” includes any step involved in the production of a polypeptide including, but not limited to, transcription, post-transcriptional modification, translation, post-translational modification, and secretion.

Germ: The “Germ” is the only living part of the corn kernel. It contains the essential genetic information, enzymes, vitamins, and minerals for the kernel to grow into a corn plant. In yellow dent corn, about 25 percent of the germ is corn oil. The endosperm covered or surrounded by the germ comprises about 82 percent of the kernel dry weight and is the source of energy (starch) and protein for the germinating seed. There are two types of endosperm, soft and hard. In the hard endosperm, starch is packed tightly together. In the soft endosperm, the starch is loose.

GH10 polypeptide: refers to a polypeptide with enzyme activity, the polypeptide being classified as member of the Glycoside hydrolase family 10 in the database of Carbohydrate-Active enZYmes (CAZymes) available at http://www.cazy.org/. (Lombard, V.; Golaconda Ramulu, H.; Drula, E.; Coutinho, P. M.; Henrissat, B. (21 Nov. 2013). “The carbohydrate-active enzymes database (CAZy) in 2013”. Nucleic Acids Research. 42 (D1): D490-D495; Cantarel B L, Coutinho P M, Rancurel C, Bernard T, Lombard V, Henrissat B (January 2009). “The Carbohydrate-Active EnZymes database (CAZy): an expert resource for Glycogenomics”. Nucleic Acids Res. 37 (Database issue): D233-8).

GH11 polypeptide refers to a polypeptide with enzyme activity, the polypeptide being classified as member of the Glycoside hydrolase family 11 in the database of Carbohydrate-Active enZYmes (CAZymes).

GH62 polypeptide: refers to a polypeptide with enzyme activity, the polypeptide being classified as member of the Glycoside hydrolase family 62 in the database of Carbohydrate-Active enZYmes (CAZymes).

Gluten: Gluten is a protein, made up from two smaller proteins, glutenin and gliadin. Herein “gluten” refers to the majority of proteins found in corn kernels. The major products of gluten from corn wet milling is corn gluten meal (Approximately 60% protein) and corn gluten feed (Approximately 20% protein).

Grind or grinding: The term “grinding” refers to breaking down the corn kernels into smaller components.

Incubation time: Time in which the one or more fractions of the corn kernel mass or the fine fiber fraction is/are in contact with hydrolytic enzyme without being subject to other processing, such as for instance screening or filtering. For example, “incubation time” may refer to the time period in which the one or more fractions of the corn kernel mass is/are in contact with enzyme during fiber washing, without being screened or subjected to other means of separation. Also, “incubation time” may refer to the time period in which a fine fiber fraction in contact with enzyme before being combined with other substrate. In many preferred embodiments, a system and method according to the present invention utilises a “space”, “void”, “incubator” or “retention tank” inside which the material is “left to be affected” by the enzymes and in such situations, the incubation time may be determined by:

$${t}_{it} = \frac{\begin{matrix}volume of incubator [ {m}^{3} ] * \\ density of inflow to incubator [ kg \text{/} {m}^{3} ]\end{matrix}}{mass inflow per time unit to the incubtaor [ kg \text{/} s ]}$$

Alternatively, if the inflow to the “space”, “void” “incubator” or “retention tank” is expressed in terms of volume per time unit:

$${t}_{it} = \frac{volume of incubator [ {m}^{3} ]}{volume inflow per time unit to the incubator [ {m}^{3} \text{/} s ]}$$

In the formulas above, “space”, “void”, “incubator” or “retention tank” are collectively referred to as “incubator”.

Mill equipment: “Mill equipment” refers to all equipment used on a mill. The wet milling process will vary dependent on the available mill equipment. Examples of mill equipment can be steeping tanks, evaporator, screw press, rotatory dryer, dewatering screen, centrifuge, hydrocyclone, ect. The size, and number of each mill equipment/milling lines can vary on different mills, which will affect the milling process. For example, the number of fiber washing screen units can vary and so can the size of a centrifuge.

Retention time: The total retention time as used herein is defined as the time period in which one or more hydrolytic enzymes are in contact with their substrate. When so stated, retention time may also refer to the time period in which the one or more hydrolytic enzymes and the substrate is retained in a specific stage or step of the wet milling process. For instance, the total retention time in the fiber washing system disclosed herein, is the time period in which the corn kernel mass, received in the first screen unit (S1) and one or more fractions thereof, are contacted with an effective amount of one or more hydrolytic enzymes before leaving the fiber washing system again. During the retention time, the one or more fractions of corn kernel mass is incubated with one or more hydrolytic enzymes in a space (V), before it leaves the fiber washing system, as part of a first fraction (s1) from the most upstream screen unit (S1) or as part of a second fraction (f4) from the most downstream screen unit (S4). Retention time may preferably be estimated as the average duration of time solid mater spends in a system according to the present invention, the system being for instance the fiber washing system or the retention tank as disclosed herein, including flow lines connecting it to other process equipment. This may be estimated by the following relation:

$${t}_{rt} = \frac{\begin{matrix}volume of system \text{:} [ {m}^{3} ] * \\ density of mass inflow [ kg \text{/} {m}^{3} ]\end{matrix}}{mass inflow per time unit to the system [ kg \text{/} s ]}$$

Alternatively, if the inflow to the system is expressed in terms of volume per time unit:

$${t}_{rt} = \frac{volume of system [ {m}^{3} ]}{volume inflow per time unit to the system [ {m}^{3} \text{/} s ]}$$

The volume of the system is typically set equal to the sum of the volumes of all voids in the system; however, as the tubing or piping in the system typically is made small, and it may thus be preferred to discard the volume of the tubing in the determination of the retention time.

Screened: The term “screened” refers to the process of separating corn kernel mass into a first fraction s and a second fraction f and movement of these fractions from one screen unit to another. A screen unit may for example be a pressure-fed screen/feed pressure screen wherein material is fed through a nozzle or a rotary screen, wherein material is forced through the screen by gravity. Examples of such screens could be DSM screen and ICM screens respectively.

A non-screening period is a non-separating period provided for incubation of corn kernel mass or fractions thereof with enzymes.

Sequence identity: The relatedness between two amino acid sequences or between two nucleotide sequences is described by the parameter “sequence identity”.

For purposes of the present invention, the degree of sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 3.0.0 or later. Version 6.1.0 was used.

The optional parameters used are gap open penalty of 10, gap extension penalty of 0.5, and the EBLOSUM62 (EMBOSS version of BLOSUM62) substitution matrix. The output of Needle labelled “longest identity” (obtained using the -nobrief option) is used as the percent identity and is calculated as follows: (Identical Residues×100)/(Length of Alignment−Total Number of Gaps in Alignment).

Starch: The term “starch” means any material comprised of complex polysaccharides of plants, composed of glucose units that occurs widely in plant tissues in the form of storage granules, consisting of amylose and amylopectin, and represented as (C₆H₁₀O₅)n, where n is any number.

Steeping or soaking: The term “steeping” means soaking the crop kernel with water and optionally SO₂.

### DESCRIPTION OF THE INVENTION

The present inventors have observed that in commercial wet milling of corn, the use of hydrolytic enzymes may significantly increase the yield of starch and gluten when contacted with a fiber fraction produced in the milling process. In particular, highly efficient separation of starch and gluten from fiber may be achieved by separating the fibers into a fine and coarse fraction and dosing the enzymes in the fine fraction. Hence, in a first aspect the invention provides a method for improving starch yield and/or gluten yield from corn kernels in a wet milling process, comprising
a) separating starch and/or gluten from fiber to provide a fiber fraction,
b) providing a fine fiber fraction by separating coarse fibers of the fiber fraction from fine fibers of the fiber fraction; and
c) contacting the fine fiber fraction with one or more hydrolytic enzymes.

The fine fiber fraction may be provided by size fractionation.

In particular embodiments of the invention, the fine fiber fraction is provided by removing fibers, which can be retained by a sieve or screen having a pore size of 1000 μm, such as by removing fibers which can be retained by a sieve or screen having a pore size of 900 μm, 800 μm, 700 μm, 600 μm or such as by removing fibers, which can be retained by a sieve or screen having a pore size of 500 μm. As a person skilled in commercial wet milling will realize, this may be achieved conveniently by dosing the enzymes in the so-called fiber press filtrate. The fiber press filtrate contains fine fiber, whereas the coarse fibers are separated out as a dry, pressed fiber fraction.

The fine fiber fraction may be provided by:
a) removing fibers which can be retained by a sieve or screen having a pore size of 1000 μm, such as a pore size of 900 μm, 800 μm, 700 μm, 600 μm or such as a pore size of 500 μm; and then
b) collecting fibers, which can be retained by a sieve or screen having a pore size of 50 μm.

The fine fiber fraction may be provided by filtration, screening, sieving, sifting, and/or application of centrifugal force.

The fine fiber fraction may be provided by passing the fiber fraction through a filter, a screen or a sieve having a pore size of 500-1000 μm, such as a pore size of 500-900 μm, 500-800 μm, 500-700 μm, or such as a pore size of 500-600 μm, and removing fibers retained by the sieve or screen. As an alternative or supplement to using a filter, sieve or screen a hydrocyclone may be used to provide the fine fiber fraction.

In further embodiments according to the invention the fine fiber fraction is provided by
a) Passing the fiber fraction through a filter, a screen or a sieve having a pore size of 500-1000 μm, such as a pore size of 500-900 μm, 500-800 μm, 500-700 μm, or such as a pore size of 500-600 μm and removing fibers retained by the filter, sieve or screen; and then
b) Passing the remaining part of the fiber fraction through a filter, a sieve or a screen having a pore size of 50 μm and collecting fibers retained by the sieve or screen.

In particular embodiments of the invention, the fine fiber fraction is provided by the use of one or more pressure fed screens and/or one or more hydrocyclones.

It is within the scope of the invention to provide for sufficient retention time in the process to allow the hydrolytic enzyme or enzymes to release residual starch bound in the fine fiber fraction. As the fine fiber fraction is usually provided in minor amounts relative to the total amount of raw material entering the process, e.g. corn kernels, a relatively long retention time may be feasible without compromising process efficiency. Hence, according to the invention, the fine fiber fraction may be contacted with said one or more hydrolytic enzymes for a time period which is from 2-72 hours, such as from 2-48 hours, form 2-24 hours, from 2-12 hours, from 2-6 hours.

As the skilled person will realize, the process of the invention may be optimized, e.g. by controlling the temperature to be within a range which encompasses the temperature optimum of the hydrolytic enzyme(s) applied. Hence, fine fibre fraction may be contacted with said one or more hydrolytic enzymes at a temperature, which is within the range of 35-70° C., such in the range of 40-60° C., such as in the range of 46-58° C., such as in the range of 47-55° C., such as in the range of 48-52° C.

In preferred embodiments of the invention, the fine fiber fraction is contacted with said one or more hydrolytic enzymes when retained in an incubator or retention tank.

The incubator or retention tank preferably provides a void of a size sufficient to provide the desired retention time, and may have means to provide appropriate process conditions during incubation of the one or more hydrolytic enzymes with the substrate. In particular, the incubator or retention tank may comprise one or more agitators configured to prevent settling of solids and/or fine fibers. It is to be understood that the present invention foresees several incubators or retention tanks coupled in series, or alternatively the use of a multi-compartment incubator or retention tank.

In some embodiments the method of the invention is one wherein starch released from the fine fiber fraction during contact with the one or more hydrolytic enzymes is separated from the fine fibers by filtration, screening, sieving, sifting, and/or application of centrifugal force.

The method according to the invention may in particular comprise the steps of:
a) soaking the corn kernels in water to produce soaked corn kernels;
b) grinding the soaked corn kernels;
c) separating germs from the ground and soaked corn kernels to produce a corn kernel mass comprising fiber, starch and gluten; and
d) subjecting the corn kernel mass, to a fiber washing procedure to separate starch and/or gluten from fiber and provide a fiber fraction.
d) separating fine fibers of said fiber fraction from coarse fibers to provide a fine fiber fraction by; and
e) contacting the fine fiber fraction with said one or more hydrolytic enzymes.

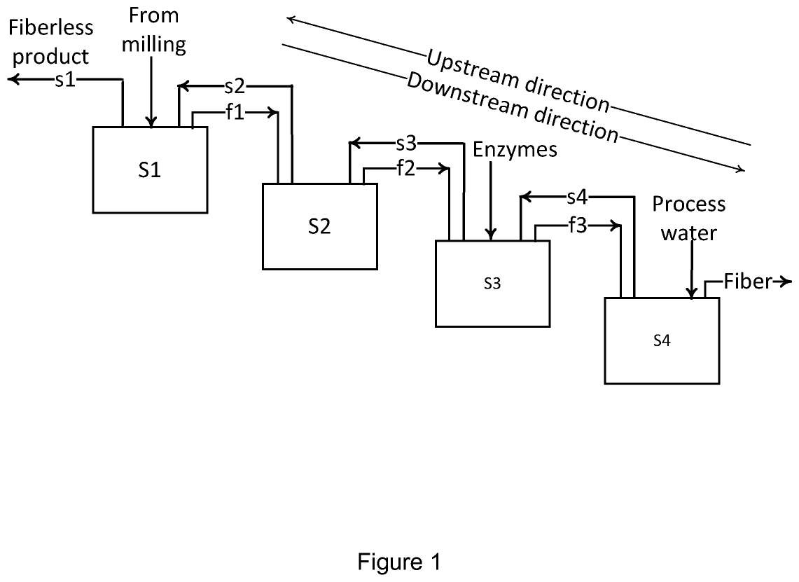

FIG. 1 schematically illustrates a fiber washing system used in particular embodiments according to the present invention. As illustrated in FIG. 1, the fiber washing system may comprise a plurality of screen units S1, S2, S3, S4 being fluently connected in a counter current washing configuration. By “fluently connected” is typically meant that the screen units or other mill equipment as disclosed herein are connected by use of flow lines, such as pipes for transporting matter between the screen units. Each of the screen units S1-S4 is configured for separating a stream of corn kernel mass and liquid into two fractions: a first fraction s (s1, s2, s3, s4) and a second fraction f (f1, f2, f3, f4). As the skilled person will understand, the number of first fractions produced in the fiber washing system depends on the number screen units included in the system. The number of screen units in the system is preferably between 2-8, and in such embodiments the number of firsts and second fractions will also be between 2-8. The screen units are typically configured so that the solid matter is separated out in a separate stream whereby the second fraction f contains a higher amount measured in wt % fiber than the first fraction s. In the figure, notation “s” preferably refers to a fibreless stream (containing starch) and notation “f” preferably refers to a fiber containing stream. Index on f and s refers to the origin of the stream. It is noted that although it is preferred that the first fractions s does not contain any fiber, this may in a practical set-up be difficult to achieve. Hence, in these embodiments the method comprises the steps of:
a) soaking the corn kernels in water to produce soaked corn kernels;
b) grinding the soaked corn kernels;
c) separating germs from the ground and soaked corn kernels to produce a corn kernel mass comprising fiber, starch and gluten;
d) providing a fiber fraction by subjecting the corn kernel mass to one or more fiber washing steps; each fiber washing step comprising passing a stream of corn kernel mass and liquid through a screen unit configured for separating the stream of corn kernel mass and liquid into two fractions: a first fraction (s) and a second fraction (f), said second fraction (f) containing a higher amount of wt % fiber than the first fraction,
e) separating fine fibers of said fiber fraction from coarse fibers to provide a fine fiber fraction by; and
f) contacting the fine fiber fraction with said one or more hydrolytic enzymes.

The method as set forth above may comprise contacting the fiber fraction and/or said second fraction (f) with one or more hydrolytic enzymes, such as one or more hydrolytic enzymes as defined below.

It is within the scope of the present invention to provide dosing of enzymes at several dosing points during the process. For instance, the process according to the invention may comprise contacting the fiber fraction and/or said fraction (f) with a first hydrolytic enzyme or a first set of hydrolytic enzymes, and contacting the fine fiber fraction with a second hydrolytic enzyme or a second set of hydrolytic enzymes; wherein the first hydrolytic enzyme or set of hydrolytic enzymes and the second hydrolytic enzyme or set of hydrolytic enzymes are the same or are different.

In currently preferred embodiments of the invention, the space (V₁), the incubator or the retention tank is in fluent connection with said fiber wash procedure, such as with a screen unit in said fiber wash procedure. This fluent connection allows for easy and convenient recycling of enzyme into the fiber washing process and for easy separation of residual starch released from the fine fiber during incubation with the one or more hydrolytic enzymes.

The flow in the fiber washing system has a downstream direction and an upstream direction: each screen unit; e.g. screen unit S3, receives a stream; e.g. f2, from an upstream screen unit, e.g. S2 and delivers a stream; e.g. s3, to the upstream screen unit; e.g. S2. Similarly, the screen unit S3 receives a stream s4 from a downstream screen unit S4 and delivers a stream f3 to the downstream screen unit S4. By adding the enzymes at an optimal point in the fiber washing system, the retention time can be prolonged, which may increase the efficiency of the removal or separation of starch from fiber. It has been found advantageous to add enzymes at a position being downstream of a most upstream screen unit 51 and upstream of a most downstream screen unit S4; in the embodiment of FIG. 1, the addition of enzymes is illustrated as being at the fluid position of the screen unit S3 (illustrated by the arrow in FIG. 1 labelled “Enzymes”. Preferably the retention time in the fiber washing system is between 35 minutes and 5 hours, such as between 45 minutes and 2.5 hours.

According to embodiments wherein the fiber washing system comprises 2 screen units, dosing is preferred between the first and second screen unit or in a space configured between screen unit 1 and screen unit 2.

According to embodiments wherein the fiber washing system comprises 3 screen units, dosing is preferred in the second screen unit or in a space configured between screen unit 1 and screen unit 3, most preferred in screen unit 2, or a space configured between screen unit 2 and 3.

According to embodiments wherein the fiber washing system comprise 4 screen units, dosing is preferred in the second or third screen unit or in a space configured between screen unit 1 and screen unit 4, most preferred in screen unit 2, or a space configured between screen unit 2 and 3.

According to embodiments wherein the fiber washing system comprise 5 screen units, dosing is preferred in the second, third or fourth screen unit, or in a space configured between screen unit 1 and screen unit 5, most preferred in screen unit 3 or a space configured between screen unit 3 and 4.

According to embodiments wherein the fiber washing system comprise 6 screen units, dosing is preferred in the second, third, fourth or fifth screen unit, or in a space configured between screen unit 1 and screen unit 6, most preferred in screen unit 4, or a space configured between screen unit 4 and 5.

According to embodiments wherein the fiber washing system comprise 7 screen units, dosing is preferred in the second, third, fourth, fifth or sixth screen unit, or in a space configured between screen unit 1 and screen unit 7, most preferred in screen unit 4 or a space configured between screen unit 4 and 5.

According to embodiments wherein the fiber washing system comprise 8 screen units, dosing is preferred in the second, third, fourth, fifth, sixth and seventh screen unit, or in a space configured between screen unit 1 and screen unit 8, most preferred in screen unit 5 or a space configured between screen unit 5 and 6.

In currently preferred embodiments of the invention the fine fiber fraction is provided as an effluent or filtrate when pressing the fiber which has been subject to fiber washing. In these embodiments the process comprises
a) feeding the fiber fraction to a fiber press (P) and subjecting it to pressing and filtration to provide pressed fiber and fiber press filtrate, and
b) feeding the fiber press filtrate to a space (V₁), and retaining the fiber press filtrate in said space (V₁) before feeding it to said fiber washing system (F).

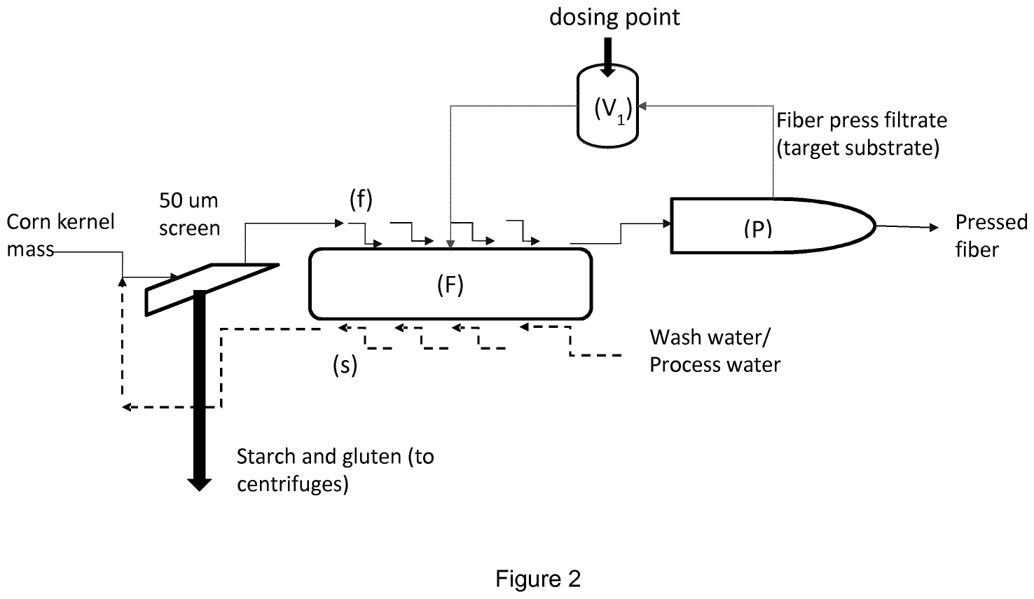

FIG. 2 schematically illustrates a process according to some embodiments of the invention. The process involves the use of a fiber washing system (F) in fluent connection with a fiber press (P), which is again connected to a space or void (V₁). The method according to the invention may hence comprise
a) providing a fiber fraction by feeding corn kernel mass to a fiber washing system (F) and subjecting it to one or more fiber washing steps; each fiber washing step comprising passing a stream of corn kernel mass and liquid through a screen unit configured for separating the stream of corn kernel mass and liquid into two fractions: a first fraction (s) and a second fraction (f), said second fraction (f) containing a higher amount of wt % fiber than the first fraction (s),
b) feeding the fiber fraction to a fiber press (P) and subjecting it to pressing and filtration to provide pressed fiber and fiber press filtrate,
c) feeding the fiber press filtrate to a space (V₁), and retaining the fiber press filtrate in said space (V₁) before feeding it to said fiber washing system (F);

wherein the fiber press filtrate is contacted with said one or more hydrolytic enzymes at a point which is between the fiber press (P) and the space (V₁), or in the space (V₁).

The incubation time in said space (V₁) may be at least 30 minutes, such as at least 1 hour, at least 2 hours, at least 4 hours, at least 6 hours, at least 8 hours or at least 10 hours, such as from 30 minutes to 48 hours, such as from 1-48 hours, 2-48 hours, 4-48 hours, 6-48 hours, 8-48 hours or such as 10-48 hours.

In these embodiments of the invention, the filtration in step b) is through a filter, a screen or a sieve having a pore size of 500-1000 μm, such as a pore size of 500-900 μm, 500-800 μm, 500-700 μm, or such as a pore size of 500-600 μm.

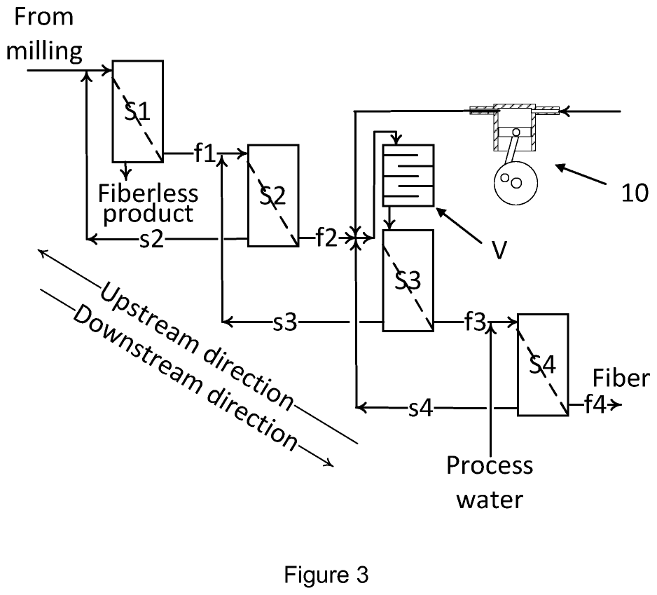

FIG. 3 schematically illustrates a fiber washing procedure in which a space (V) is arranged between the screening units of the system. As illustrated, in the method according to the invention, the fiber washing procedure may be performed with the use of a fiber washing system comprising
i) a plurality of screen units (S1 . . . S4) being fluently connected in a counter current washing configuration; each screen unit is configured for separating a stream of corn kernel mass and liquid into two fractions:
   a first fraction (s) and
   a second fraction (f);
   said second fraction (f) containing a higher amount measured in wt % fiber than the first fraction (s);
ii) optionally a space (V) arranged in the system and being fluently connected to receive one of said first fraction (s), one of said second fraction (f), or a mixed first and second fraction (s,f), preferably only a second fraction (f), and configured to provide an incubation time for one or both fractions received in the space; and outletting the thereby incubated one or both fractions to a downstream screen unit (S4),

wherein the system is configured for
inletting corn kernel mass and liquid to the most upstream screen unit (S1)
outletting the first fraction (s1) from the most upstream screen unit (S1) as a product stream containing starch,
inletting process water, preferably arranged for inletting process water to a most downstream screen unit (S4),
outletting the second fraction (f4) from most downstream screen unit (S4) as a washed corn kernel mass containing a lower amount of starch and gluten than the original corn kernel mass; and
optionally introducing hydrolytic enzymes into the system.

The one or more hydrolytic enzymes may be dosed in amounts corresponding to 5-500 g enzyme protein (EP), such as 5-300 g EP/metric tonne corn kernels, 5-200 g EP/metric tonne corn kernels, 5-100 g EP/metric tonne corn kernels, 10-500 g EP/metric tonne corn kernels, 10-300 g EP/metric tonne corn kernels, 10-200 g EP/metric tonne corn kernels or such as 10-100 g EP/metric tonne corn kernels. The one or more hydrolytic enzymes may also be dosed in amounts corresponding to 0.2-15 mg enzyme protein (EP)/g fiber, such as 0.2-10 mg EP/g fiber, 0.2-5 mg EP/g fiber, 0.4-15 mg EP/g fiber, 0.4-10 mg EP/g fiber, or such as 0.4-5 mg EP/g fiber.

The incubation time in said space (V) may be in the range of 0.5-3 hours, such as 1-3 hours, 1-2 hours or such as 85-95 minutes. The space (V) may have a volume which is in the range of 50-1000 m³, such as in the range of 50-500 m³, 100-1000 m³, 100-500 m³, 200-1000 m³, 200-500 m³, or 500-1000 m³.

The one or more hydrolytic enzymes may be selected from the group consisting of cellulases (EC 3.2.1.4), xylanases (EC 3.2.1.8) arabinofuranosidases (EC 3.2.1.55 (Non-reducing end alpha-L-arabinofuranosidases); EC 3.2.1.185 (Non-reducing end beta-L-arabinofuranosidases) cellobiohydrolase I (EC 3.2.1.150), cellobiohydrolase II (E.C. 3.2.1.91), cellobiosidase (E.C. 3.2.1.176), beta-glucosidase (E.C. 3.2.1.21), beta-xylosidases (EC 3.2.1.37).

One or more of said hydrolytic enzymes may be expressed in an organism with a cellulase background, such as Trichoderma reesei.

The one or more hydrolytic enzymes may comprise a GH10 polypeptide with xylanase activity and/or a GH11 polypeptide with xylanase activity.

The one or more hydrolytic enzymes may comprise a GH61 polypeptide with arabinofuranosidase activity and/or a GH62 polypeptide with arabinofuranosidase activity.

The one or more hydrolytic enzymes may in particular comprise a GH62 polypeptide with arabinofuranosidase activity, which is selected from the group consisting of:
i) An amino acid sequence as set forth in any one of SEQ ID NOs: 1-21
ii) An amino acid sequence which has at least 80% sequence identity to any one of the amino acid sequences set forth in SEQ ID NO:1-21; and
iii) A subsequence of any one of the amino acid sequences in iv) and v).

The one or more hydrolytic enzymes may comprise a GH10 polypeptide with xylanase activity, which is selected from the group consisting of
i) An amino acid sequence as set forth in any one of SEQ ID NOs: 22-26
ii) An amino acid sequence which has at least 80% sequence identity to any one of SEQ ID NOs: 22-26; and
iii) A subsequence of any one of the amino acid sequences in i) and ii).

The one or more of said hydrolytic enzymes may comprise a GH11 polypeptide with xylanase activity, which is selected from the group consisting of
i) An amino acid sequence as set forth in any one of SEQ ID NOs: 27-35
ii) An amino acid sequence which has at least 80% identity to any one of SEQ ID NOs: 27-35; and
iii) A subsequence of any one of the amino acid sequences in i) and ii).

The one or more hydrolytic enzymes may be expressed in Trichoderma reesei and comprise a xylanase, which is a GH10 xylanase and a arabinofuranosidase, which is a GH62 arabinofuranosidase.

In currently preferred embodiments, the process uses Frontia®Fiberwash, a commercial product comprising a GH10 xylanase and a GH62 arabinofuranosidase, which is commercially available from Novozymes A/S, Bagsvaerd, Denmark

Referring to the schematic representation in FIG. 2, the method of the invention may comprise
a) providing a fiber fraction by feeding corn kernel mass to a fiber washing system (F) and subjecting it to one or more fiber washing steps; each fiber washing step comprising passing a stream of corn kernel mass and liquid through a screen unit configured for separating the stream of corn kernel mass and liquid into two fractions: a first fraction (s) and a second fraction (f), said second fraction (f) containing a higher amount of wt % fiber than the first fraction (s),
b) feeding the fiber fraction to a fiber press (P) and subjecting it to pressing and filtration to provide pressed fiber and fiber press filtrate or fine fiber fraction,
c) feeding the fiber press filtrate or fine fiber fraction to a space (V₁), and retaining the fiber press filtrate or fine fiber fraction in said sp ace (V₁) before feeding it to said fiber washing system (F);

wherein the fiber press filtrate or fine fiber fraction is contacted with said one or more hydrolytic enzymes by dosing the enzyme(s) at a point, which is between the fiber press (P) and the space (V₁), or in the space (V₁).

A second aspect of the invention provides a corn kernel wet milling system comprising
i) a fiber washing system (F)
ii) a fiber press (P),
iii) means for dosing one or more hydrolytic enzymes; and
iv) a space (V₁);

wherein said fiber washing system (F), said fiber press (P) and said space (V₁) are interconnected to allow a flow of a fiber fraction from the fiber washing system to the press, and a flow of fiber press filtrate or a fine fiber fraction from the press to the space (V₁); wherein the said means for dosing one or more hydrolytic enzymes is configured for dosing, said enzymes to the fiber press filtrate or the fine fiber fraction, and said space (V₁) has a volume of at least 100 m³/1000 tonne corn processed per day.

As the skilled person will realize in relation to the method and the milling system provided herein, the required volume of said space (V₁) depends to some extent on the configuration of the particular wet mill and the process parameters chosen in the wet milling process. Relevant process parameters include the relative amount of dry substance DS for incubation, the flowrate of the fiber press filtrate or fine fiber fraction and the desired incubation time. For example for a plant processing at 1000 Tonne corn per day, operating with an amount of dry substance in the fiber press filtrate or fine fiber fraction corresponding to 4% DS and 8 hour incubation time, the ideal volume of the space (V₁) is 80 to 100 m³. Hence in further embodiments, the space (V₁) has a volume of 50 and 250 m³ per 1000 Tonne of corn processed a day, such as a volume of 50 and 250 m³ per 1000 Tonne of corn processed a day. In absolute numbers, the preferred volume of the space (V₁) is 50 and 500 m³, such as 50 and 400 m³, 50 and 300 m³, 50 and 250 m³, 80 and 500 m³, 80 and 400 m³, or 80 and 250 m³.

The said means for dosing one or more hydrolytic enzymes (a dosing device) is/are included in the schematic representation in FIG. 3. The dosing device (10) is typically adapted to provide a controllable dosing quantity of enzymes, preferably according to a predetermined specific ratio between amount of enzymes and infeed of corn kernel mass to the system. To accomplish this, the dosing device could be a metering pump as illustrated by a piston pump in FIG. 3.

The corn kernel wet milling system according to the invention may be a milling system in which
said fiber washing system (F), said fiber press (P) and said space (V₁) are interconnected to allow a flow of a fiber fraction from the fiber washing system to the press, a flow of fiber press filtrate or fine fiber fraction from the press to the space (V₁) and a flow of fiber press filtrate or fine fiber fraction from the space (V₁) to the fiber washing system;
and
said space (V₁) is arranged at a position, which is between the fiber press and the fiber washing system.

The corn kernel wet milling system according to the invention may comprise a screen unit or set of screen units connected to or integrated in the press, the screen unit or set of screen units being configured to produce a fine fiber fraction as defined according to the invention.

In particular embodiments, the space (V₁) is connected to the fiber washing system (F) so as to allow the fiber press filtrate or the fine fiber fraction to flow from the space (V₁) into the fiber wash system (F).

The corn kernel wet milling system according to the invention may in particular comprise a fiber washing system comprising
i) a plurality of screen units (S1 . . . S4) being fluently connected in a counter current washing configuration; each screen unit being configured for separating a stream of corn kernel mass and liquid into two fractions:
   a first fraction (s) and
   a second fraction (f);
   said second fraction (f) containing a higher amount measured in wt % fiber than the first fraction (s);
ii) optionally a space (V) arranged in the system and being fluently connected to receive one of said first fraction (s), one of said second fraction (f), or a mixed first and second fraction (s,f), preferably only a second fraction (f), and configured to provide an incubation time for one or both fractions received in the space; and outletting the thereby incubated one or both fractions to a downstream screen unit (S4),

wherein the system is configured for
inletting corn kernel mass and liquid to the most upstream screen unit (S1)
outletting the first fraction (s1) from the most upstream screen unit (S1) as a product stream containing starch,
inletting process water, preferably arranged for inletting process water to a most downstream screen unit (S4),
outletting the second fraction (f4) from most downstream screen unit (S4) as a washed corn kernel mass containing a lower amount of starch and gluten than the original corn kernel mass; and
optionally introducing hydrolytic enzymes into the system.

The corn kernel wet milling system according to the invention may further comprise
i) a grinding or milling device configured for grinding or milling of steeped crop kernels,
ii) a first screen unit or set of screen units configured to receive the ground or milled steeped crop kernels and to separate germ from the starch, gluten and fiber,
iii) a second set of screen units configured in a fiber washing system (F) to receive the starch, gluten and fiber and separate the fiber from the starch and gluten,
iv) a press (P),
v) a third screen unit or set of screen units configured to produce a fine fiber fraction, such as fine fiber fraction defined above;
vi) an enzyme dosing system configured to contact the fine fiber fraction with one or more hydrolytic enzymes, and
vii) a space (V₁);
   wherein the space (V₁) is fluently connected to said third screen unit or set of screen units to receive and retain the fine fiber fraction and enzyme(s), and is fluently connected to said fiber washing system to deliver the fine fiber fraction and enzyme(s) to the fiber washing system.

In a final aspect the present invention provides a fiber fraction, such as a fine fiber fraction, which is obtainable or is obtained by the method according to the invention.

Although the present invention has been described in connection with the specified embodiments, it should not be construed as being in any way limited to the presented examples. The scope of the present invention is set out by the accompanying claim set. In the context of the claims, the terms “comprising” or “comprises” do not exclude other possible elements or steps. Also, the mentioning of references such as “a” or “an” etc. should not be construed as excluding a plurality. The use of reference signs in the claims with respect to elements indicated in the figures shall also not be construed as limiting the scope of the invention. Furthermore, individual features mentioned in different claims, may possibly be advantageously combined, and the mentioning of these features in different claims does not exclude that a combination of features is not possible and advantageous.

### Embodiments

The invention can also be described by the following embodiments
1. A method for improving starch yield and/or gluten yield from corn kernels in a wet milling process, comprising
   a) separating starch and/or gluten from fiber to provide a fiber fraction,
   b) providing a fine fiber fraction by separating coarse fibers of the fiber fraction from fine fibers of the fiber fraction; and
   c) contacting the fine fiber fraction with one or more hydrolytic enzymes.
2. The method according to embodiment 1, wherein the fine fiber fraction is provided by size fractionation.
3. The method according to any of the preceding embodiments, wherein the fine fiber fraction is provided by removing fibers, which can be retained by a filter, sieve or screen having a pore size of 1000 μm.
4. The method according to any of the preceding embodiments, wherein the fine fiber fraction is provided by:
   a) removing fibers which can be retained by a filter, sieve or screen having a pore size of 1000 μm; and then
   b) collecting fibers, which can be retained by a filter, sieve or screen having a pore size of 50 μm.
5. The method according to any of the preceding embodiments, wherein the fine fiber fraction is provided by filtration, screening, sieving, sifting, and/or application of centrifugal force.
6. The method according to any of the preceding embodiments, wherein the fine fiber fraction is provided by passing the fiber fraction through a filter, a screen or a sieve having a pore size of 1000 μm and removing fibers retained by the sieve or screen
7. The method according to any of the preceding embodiments, wherein the fine fiber fraction is provided by
   a) Passing the fiber fraction through a filter, a screen or a sieve having a pore size of 1000 μm and removing fibers retained by the filter, sieve or screen; and then
   b) Passing the remaining part of the fiber fraction through a filter, a sieve or a screen having a pore size of 50 μm and collecting fibers retained by the sieve or screen.
8. The method according to any of the preceding embodiments, wherein the fine fiber fraction is provided by the use of one or more pressure fed screens and/or one or more hydrocyclones.
9. The method according to any of the preceding embodiments, wherein the fine fiber fraction is contacted with said one or more hydrolytic enzymes for a time period which is from 2-72 hours.
10. The method according to any of the preceding embodiments, wherein the fine fiber fraction is contacted with said one or more hydrolytic enzymes at a temperature which is within the range of 35-70° C., such in the range of 40-60° C., such as in the range of 46-58° C., such as in the range of 47-55° C., such as in the range of 48-52° C.
11. The method according to any of the preceding embodiments, wherein the fine fiber fraction is contacted with said one or more hydrolytic enzymes when retained in an incubator or retention tank.
12. The method according to any of the preceding embodiments, wherein said the incubator or retention tank comprises one or more agitators configured to prevent settling of solids and/or fine fibers.
13. The method according to any of the preceding embodiments, wherein starch released from the fine fiber fraction during contact with the one or more hydrolytic enzymes is separated from the fine fibers by filtration, screening, sieving, sifting, and/or application of centrifugal force.
14. The method according to any of the preceding embodiments, comprising the steps of:
   a) soaking the corn kernels in water to produce soaked corn kernels;
   b) grinding the soaked corn kernels;
   c) separating germs from the ground and soaked corn kernels to produce a corn kernel mass comprising fiber, starch and gluten; and
   d) subjecting the corn kernel mass, to a fiber washing procedure to separate starch and/or gluten from fiber and provide a fiber fraction.
   d) separating fine fibers of said fiber fraction from coarse fibers to provide a fine fiber fraction by; and
   e) contacting the fine fiber fraction with said one or more hydrolytic enzymes.
15. The method according to any of the preceding embodiments, comprising the steps of:
   a) soaking the corn kernels in water to produce soaked corn kernels;
   b) grinding the soaked corn kernels;
   c) separating germs from the ground and soaked corn kernels to produce a corn kernel mass comprising fiber, starch and gluten;
   d) providing a fiber fraction by subjecting the corn kernel mass to one or more fiber washing steps; each fiber washing step comprising passing a stream of corn kernel mass and liquid through a screen unit configured for separating the stream of corn kernel mass and liquid into two fractions: a first fraction (s) and a second fraction (f), said second fraction (f) containing a higher amount of wt % fiber than the first fraction,
   e) separating fine fibers of said fiber fraction from coarse fibers to provide a fine fiber fraction by; and
   f) contacting the fine fiber fraction with said one or more hydrolytic enzymes.
16. The method according to embodiment 15, comprising contacting the fiber fraction and/or said second fraction (f) with one or more hydrolytic enzymes.
17. The method according to embodiment 15 or 16, comprising contacting the fiber fraction and/or said fraction (f) with a first hydrolytic enzyme or a first set of hydrolytic enzymes, and contacting the fine fiber fraction with a second hydrolytic enzyme or a second set of hydrolytic enzymes; wherein the first hydrolytic enzyme or set of hydrolytic enzymes and the second hydrolytic enzyme or set of hydrolytic enzymes are the same or are different.
18. The method according to any of embodiments 11-17, wherein said incubator or retention tank is in fluent connection with a screen unit in said fiber wash procedure.
19. The method according to any of the preceding embodiments, comprising
   a) feeding the fiber fraction to a fiber press (P) and subjecting it to pressing and filtration to provide pressed fiber and fiber press filtrate, and
   b) feeding the fiber press filtrate to a space (V₁), and retaining the fiber press filtrate in said space (V₁) before feeding it to said fiber washing system (F);
20. The method according to any of the preceding embodiments, comprising
   a) providing a fiber fraction by feeding corn kernel mass to a fiber washing system (F) and subjecting it to one or more fiber washing steps; each fiber washing step comprising passing a stream of corn kernel mass and liquid through a screen unit configured for separating the stream of corn kernel mass and liquid into two fractions: a first fraction (s) and a second fraction (f), said second fraction (f) containing a higher amount of wt % fiber than the first fraction (s),
   b) feeding the fiber fraction to a fiber press (P) and subjecting it to pressing and filtration to provide pressed fiber and fiber press filtrate,
   c) feeding the fiber press filtrate to a space (V₁), and retaining the fiber press filtrate in said space (V₁) before feeding it to said fiber washing system (F);
   wherein the fiber press filtrate is contacted with said one or more hydrolytic enzymes at a point which is between the fiber press (P) and the space (V₁), or in the space (V₁)
21. The method according to embodiment 20, wherein the filtration in step b) is through a filter, a screen or a sieve having a pore size of 1000 μm.
22. The method according to embodiment 20 or 21, wherein the incubation time in said space (V₁) is at least 30 minutes, such as from 30 minutes to 48 hours.
23. The method according to any of the preceding embodiments, wherein said fiber washing procedure is performed with the use of a fiber washing system comprising
   i) a plurality of screen units (S1 . . . S4) being fluently connected in a counter current washing configuration; each screen unit being configured for separating a stream of corn kernel mass and liquid into two fractions:
      a first fraction (s) and
      a second fraction (f);
      said second fraction (f) containing a higher amount measured in wt % fiber than the first fraction (s);
   ii) optionally a space (V) arranged in the system and being fluently connected to receive one of said first fraction (s), one of said second fraction (f), or a mixed first and second fraction (s,f), preferably only a second fraction (f), and configured to provide an incubation time for one or both fractions received in the space; and outletting the thereby incubated one or both fractions to a downstream screen unit (S4),
   wherein the system is configured for
      inletting corn kernel mass and liquid to the most upstream screen unit (S1)
      outletting the first fraction (s1) from the most upstream screen unit (S1) as a product stream containing starch,
      inletting process water, preferably arranged for inletting process water to a most downstream screen unit (S4),
      outletting the second fraction (f4) from most downstream screen unit (S4) as a washed corn kernel mass containing a lower amount of starch and gluten than the original corn kernel mass; and
      optionally introducing hydrolytic enzymes into the system.
24. The method according to embodiment 23, wherein said space (V) has a volume which is in the range of 50-1000 m³.
25. The method according to any of embodiments 19-24, wherein said space (V₁) has a volume which is in the range of 80 and 250 m³.
26. The method according to any of the preceding embodiments, wherein the one or more hydrolytic enzymes is/are dosed in amounts corresponding to 5-500 g enzyme protein (EP)/metric tonne corn kernels, such as 5-300 g EP/metric tonne corn kernels, 5-200 g EP/metric tonne corn kernels, 5-100 g EP/metric tonne corn kernels, 10-500 g EP/metric tonne corn kernels, 10-300 g EP/metric tonne corn kernels, 10-200 g EP/metric tonne corn kernels or such as 10-100 g EP/metric tonne corn kernels or is/are dosed in amounts corresponding to 0.2-15 mg enzyme protein (EP)/g fiber, such as 0.2-10 mg EP/g fiber, 0.2-5 mg EP/g fiber, 0.4-15 mg EP/g fiber, 0.4-10 mg EP/g fiber, or such as 0.4-5 mg EP/g fiber.
27. The method according to any of the preceding embodiments, wherein said one or more hydrolytic enzymes is/are selected from the group consisting of cellulases (EC 3.2.1.4), xylanases (EC 3.2.1.8) arabinofuranosidases (EC 3.2.1.55 (Non-reducing end alpha-L-arabinofuranosidases); EC 3.2.1.185 (Non-reducing end beta-L-arabinofuranosidases) cellobiohydrolase I (EC 3.2.1.150), cellobiohydrolase II (E.C. 3.2.1.91), cellobiosidase (E.C. 3.2.1.176), beta-glucosidase (E.C. 3.2.1.21), beta-xylosidases (EC 3.2.1.37).
28. The method according to any of the preceding embodiments, wherein one or more of said hydrolytic enzymes is/are expressed in an organism with a cellulase background, such as Trichoderma reesei.
29. The method according to any of the preceding embodiments, wherein the one or more hydrolytic enzymes comprise a GH10 polypeptide with xylanase activity and/or a GH11 polypeptide with xylanase activity.
30. The method according to any of the preceding embodiments, wherein the one or more hydrolytic enzymes comprise a GH61 polypeptide with arabinofuranosidase activity and/or a GH62 polypeptide with arabinofuranosidase activity.
31. The method according to any of the preceding embodiments, wherein the one or more hydrolytic enzymes comprise a GH62 polypeptide with arabinofuranosidase activity, which is selected from the group consisting of:
   i) An amino acid sequence as set forth in any one of SEQ ID NOs: 1-21
   ii) An amino acid sequence which has at least 80% identity to any one of the amino acid sequences set forth in SEQ ID NO:1-21; and
   iii) A subsequence of any one of the amino acid sequences in iv) and v).
32. The method according to any of the preceding embodiments 1-31, wherein the one or more hydrolytic enzymes comprise a GH10 polypeptide with xylanase activity, which is selected from the group consisting of
   i) An amino acid sequence as set forth in any one of SEQ ID NOs: 22-26
   ii) An amino acid sequence which has at least 80% identity to any one of SEQ ID NOs: 22-26; and
   iii) A subsequence of any one of the amino acid sequences in i) and ii).
33. The method according to any of embodiments 1-32, wherein the one or more of said hydrolytic enzymes comprise a GH11 polypeptide with xylanase activity, which is selected from the group consisting of
   i) An amino acid sequence as set forth in any one of SEQ ID NOs: 27-35
   ii) An amino acid sequence which has at least 80% identity to any one of SEQ ID NOs: 27-35; and
   iii) A subsequence of any one of the amino acid sequences in i) and ii).
34. The method according to any of the preceding embodiments, wherein the one or more hydrolytic enzymes is expressed in Trichoderma reesei and comprise a xylanase, which is a GH10 xylanase and a arabinofuranosidase, which is a GH62 arabinofuranosidase.
35. The method according to any of the preceding embodiments, comprising
   a) providing a fiber fraction by feeding corn kernel mass to a fiber washing system (F) and subjecting it to one or more fiber washing steps; each fiber washing step comprising passing a stream of corn kernel mass and liquid through a screen unit configured for separating the stream of corn kernel mass and liquid into two fractions: a first fraction (s) and a second fraction (f), said second fraction (f) containing a higher amount of wt % fiber than the first fraction (s),
   b) feeding the fiber fraction to a fiber press (P) and subjecting it to pressing and filtration to provide pressed fiber or coarse fiber fraction and fiber press filtrate or fine fiber fraction,
   c) feeding the fiber press filtrate or fine fiber fraction to a space (V₁), and retaining the fiber press filtrate or fine fiber fraction in said space (V₁) before feeding it to said fiber washing system (F);

wherein the fiber press filtrate or fine fiber fraction is contacted with said one or more hydrolytic enzymes at a point which is between the fiber press (P) and the space (V₁), or in the space (V₁).
36. A corn kernel wet milling system comprising
   i) a fiber washing system (F)
   ii) a fiber press (P),
   iii) means for dosing one or more hydrolytic enzymes; and
   iv) a space (V₁);
   wherein said fiber washing system (F), said fiber press (P) and said space (V₁) are interconnected to allow a flow of a fiber fraction from the fiber washing system to the press, a flow of fiber press filtrate or a fine fiber fraction from the press to the space (V₁);
   wherein the said means for dosing one or more hydrolytic enzymes is configured for dosing said enzymes to the fiber press filtrate or the fine fiber fraction
   and said space (V₁) has a volume of at least 100 m³/1000 tonne corn processed per day.
37. The corn kernel wet milling system according to embodiment 36, wherein
   said fiber washing system (F), said fiber press (P) and said space (V₁) are interconnected to allow a flow of a fiber fraction from the fiber washing system to the press, a flow of fiber press filtrate or fine fiber fraction from the press to the space (V₁) and a flow of fiber press filtrate or fine fiber fraction from the space (V₁) to the fiber washing system;
   and
   said space (V₁) is arranged at a position, which is between the fiber press and the fiber washing system.
38. The corn kernel wet milling system according to embodiment 36-37, comprising a screen unit or set of screen units connected to or integrated in the press, the screen unit or set of screen units being configured to produce a fine fiber fraction as defined in any of embodiments 2-8.
39. The corn kernel wet milling system according to embodiment 36-38, wherein the space (V₁) is connected to the fiber was system (F) so as to allow the fiber press filtrate or the fine fiber fraction to flow from the space (V₁) into the fiber wash system (F).
40. The corn kernel wet milling system according to any of embodiment 36-39, wherein the fiber washing system is as defined in embodiment 23.
41. The corn kernel wet milling system according to any of embodiments 36-40, comprising
   i) a grinding or milling device configured for grinding or milling of steeped crop kernels,
   ii) a first screen unit or set of screen units configured to receive the ground or milled steeped crop kernels and to separate germ from the starch, gluten and fiber,
   iii) a second set of screen units configured in a fiber washing system (F) to receive the starch, gluten and fiber and separate the fiber from the starch and gluten,
   iv) a press (P),
   v) a third screen unit or set of screen units configured to produce a fine fiber fraction as defined in any of embodiments 2-8;
   vi) an enzyme dosing system configured to contact the fine fiber fraction with one or more hydrolytic enzymes, and
   vii) a space (V₁);
   wherein the space (V₁) is fluently connected to said third screen unit or set of screen units to receive and retain the fine fiber fraction and enzyme(s), and is fluently connected to said fiber washing system to deliver the fine fiber fraction and enzyme(s) to the fiber washing system.
42. A fiber fraction, such as a fine fiber fraction, which is obtainable or is obtained by the method according to any of embodiments 1-34.

### EXAMPLES

### Example 1

In this example, we measured the insoluble solids that were recovered from fine fiber, residual starch in fine fiber and residual protein in fine fiber after incubation with enzyme at varying doses and incubation times.

The fine fiber sample was obtained from a wet-mill plant with a total dry matter content of 5.7%. The sample was re-suspended in tap water to a slurry containing dry solids 4%. To this slurry FrontieFiberWash (commercially available from Novozymes NS, Bagsvaerd, Denmark) was added at 10.59 mg product/g fiber and 31.76 mg product/g fiber.

10.59 mg product/g fiber dose assumes the 3× recycle effect of the industrial process: The recycle effect is due to the counter current flow of streams in the wet milling process and recycling of process water, and is achieved when the wet mill is operated at equilibrium.

0.3 kg/MT wet corn*3 (recycle effect)/85% Dry solids of corn/10% fiber in corn.

10.59 mg product/g fiber*fine fiber dry solids=enzyme dose

The % DS was adjusted with amounts of water as shown in Table 1 below to achieve 4% DS.

(Fiber dry solids/% dried solids)−Fiber weight=Water added

TABLE 1

| | | | | | | Total | |
|---|---|---|---|---|---|---|---|
| Time | | Fiber | | | | vol. | Water |
| incubated | Tube | weight | Fiber | Frontia ® | | add | added |
| (min) | # | (g) | DS (g) | Fiberwash | Buffer | (ml) | (ml) |
| 0 | 1 | 44.93 | 2.56 | 0 | 0.0 | 0.00 | 19.2 |
| 0 | 2 | 44.07 | 2.51 | 0 | 0.0 | 0.00 | 19.2 |
| 2 | 3 | 44.48 | 2.54 | 201 | 0.0 | 0.20 | 19.0 |
| 2 | 4 | 44.29 | 2.52 | 200 | 0.0 | 0.20 | 19.0 |
| 4 | 5 | 44.19 | 2.52 | 67 | 0.0 | 0.07 | 19.2 |
| 4 | 6 | 44.46 | 2.53 | 67 | 0.0 | 0.07 | 19.2 |
| 24 | 7 | 44.22 | 2.52 | 67 | 0.0 | 0.07 | 19.2 |
| 24 | 8 | 44.56 | 2.54 | 67 | 0.0 | 0.07 | 19.2 |

The incubation was done at 50° C. in a Thermo Fisher air shaker with constant mixing for various times 0, 2, 4 and 24 hours. After incubation, the samples were cooled quickly in ice-water (5° C.) before processing.

The slurry was then poured on to a 50 μm screen and a catch pan. The filtrate was caught with catch pan. The insoluble solids in the catch pan were transferred into a 500 ml Nalgene bottle. The fine fiber was scraped off the 50 μm screen and washed with 200 mls of DI water in a beaker with a spatula. The fine fiber slurry was then poured over a 50 μm sieve and catch pan, the insoluble solids were transferred to the same Nalgene bottle.

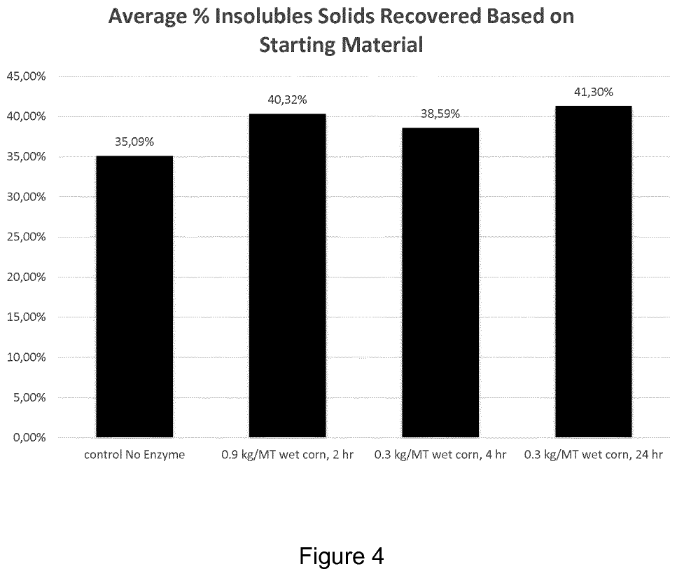

The Nalgene bottle was then capped and the insoluble solids were separated using vacuum filtration. The vacuum filtration set up utilized a funnel with filter paper (Whatman) the insoluble solids slurry was poured over the filter paper under vacuum. A weight was taken of the filter paper before filtration and filter paper was put in a 50° C. oven to dry and a weight was taken after 24 hours in the oven. The insoluble solids are reported in Table 2 below and in FIG. 4.

TABLE 2

| Industrial Dose | % insolubles released from | Incubation |
|---|---|---|
| kg/mt wet corn | starting Fine Fiber | Time (Hrs) |
| 0 | 35% | 0 |
| 0 | 35% | 0 |
| 0.9 | 39% | 2 |
| 0.9 | 42% | 2 |
| 0.3 | 38% | 4 |
| 0.3 | 39% | 4 |
| 0.3 | 40% | 24 |
| 0.3 | 42% | 24 |

The effect of Frontia®Fiberwash is apparent from the quantity of insoluble solids generated over the range of doses and incubation times.

The fine fiber left on the 50 μm sieve was scooped off the sieve and placed in a 50° C. oven overnight. After drying overnight in a 50° C. oven the residual fine fiber was placed in a 105° C. oven for 1 hour to remove any residual water. An amount of the residual fine fiber was weighed and placed in an amber bottle with 50 mls of 0.4M HCl. The bottles were loosely capped and swirled. The bottles were placed in an autoclave for 80 minutes at 230 F. After 80 minutes the bottles were cooled to room temp and filtered through a 0.45 μm filter into HPLC vials.

HPLC analysis was carried out with an Agilent 1200 HPLC.

| | |
|---|---|
| Degasser | Model #: G4225A Serial #: |
| | JPAAA04141 |
| Pump | Model #: G1310B Serial #: |
| | DEAB903158 |
| Auto | Model #: G1367E Serial #: |
| Sampler | DEAC00461 |
| Thermostatted | Model #: G1330B Serial #: |
| Auto | DEBAK18815 |
| Column | Model #: G1316A Serial #: |
| Compartmen | DEACN28754 |
| Detector 1 | Model #: G1362A Serial #: |
| Refractive | DEAA604993 |

Column: HPX-87H column and guard column
Flow rate: 0.60 ml/min.
Mobile phase: 0.005M H₂SO₄
Temp: 65° C. (column temperature)
Injection volume: 10.0 ul
Run Time: 30 minutes
Detection: Refractive Index (RI) @ 50° C.

Calculation

$$Total Volume ( mls ) = volume 0.4 M HCl ( mls ) + ( 1 - \% DS \text{/} 100 ) \times weight ( g )$$

$$Total Glucose ( g ) = ( \% w \text{/} v glucose ( g \text{/} ml ) * total volume ( mls ) ) \text{/} 100$$

$$Total Starch ( g ) = \frac{total glucose ( g )}{1.111}$$

$$NOTE \text{:} 1.111 is the starch hydration factor . \\ \% Starch = \frac{total starch ( g )}{weight ( g ) \times \% DS \text{/} 100}$$

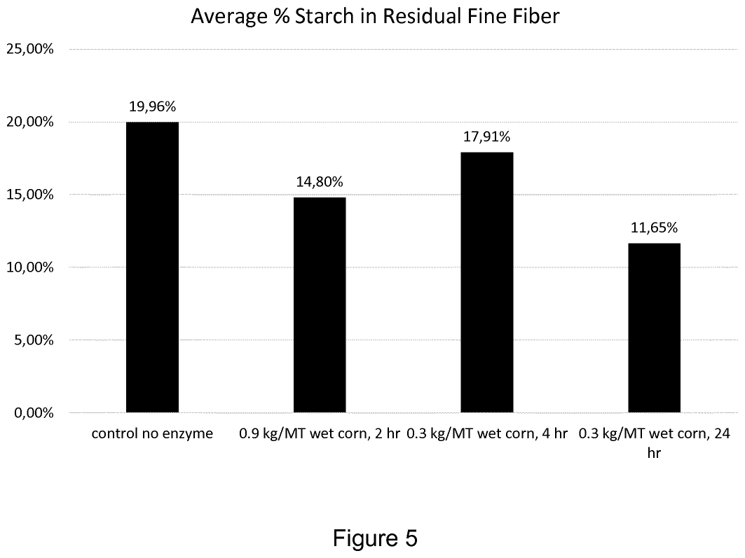

The results (average % (w/w) starch in residual fine fiber) are shown in FIG. 5.

The effect of Frontia®FiberWash is apparent from the percentage of starch removed from the various enzyme treated fine fiber samples over the range of doses and incubation times.

100 mg of dried residual fine fiber was run on a LECO FP-628 Nitrogen Analyzer. The LECO Nitrogen Analyzer CHN628 Series Elemental Determinator is used to determine nitrogen, carbon/nitrogen, and carbon/hydrogen/nitrogen in organic matrices. The instrument utilizes a combustion technique and provides a result within 4.5 minutes for all the elements being determined. The instrument features custom Windows®-based software operated through an external PC to control the system operation and data management.

A pre-weighed and encapsulated sample was placed in the instrument's loader where the sample will be transferred to the instrument's purge chamber directly above the furnace, eliminating the atmospheric gases from the transfer process. The sample is then introduced to the primary furnace containing only pure oxygen, resulting in a rapid and complete combustion (oxidation) of the sample. Carbon, hydrogen, and nitrogen present in the sample are oxidized to carbon dioxide (CO₂), water (H₂0), and NOx respectively, and are swept by the oxygen carrier through a secondary furnace for further oxidation and particulate removal. In the FP and CN628 models, the combustion gases pass through a pre-cooler and thermoelectric cooler to remove the water vapor. The combination gases are then collected in a vessel known as a ballast for equilibration. The homogenized gases from the ballast are swept through a 10 cc aliquot loop and then passed into a carrier gas. Separate optimized non-dispersive infrared (NDIR) cells are utilized for the detection of H₂O and CO₂, ensuring the rapid analysis time of the system. The NOx gases are passed through a reduction tube filled with copper to reduce the gases to N₂ and remove any excess oxygen present from the combustion process. The aliquot gas then passes through LECOSORB and Anhydrone to remove CO₂ and the water generated during the CO₂ trapping process and onto a thermal conductivity cell (TC) utilized to detect the N₂.

The final results are typically displayed in weight percent or parts-per-million but can be displayed in other custom units or conversions such as percent total protein, moisture corrected, and others.

The LECO FP-628 was set up with
Steel wool Furnace filter tube
Anhydrone Furnace filter tube
Porous Crucible
psi Oxygen
psi Helium
6.25 Corn Protein Factor based on the user manual average protein contains 16% nitrogen=100/16=6.25

The percent nitrogen was measured based on the mass of the sample.

% Nitrogen of sample*6.25(corn protein factor)=Protein (%)

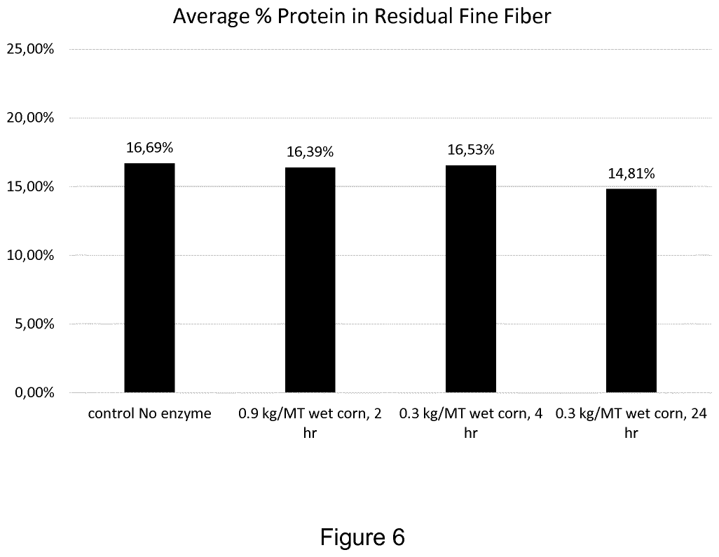

The results are shown in FIG. 6. The effect of Frontia®Fiberwash is apparent from the percentage of protein removed from the various enzyme treated fine fiber samples over the range of doses and incubation times.

1. A method for improving starch yield and/or gluten yield from corn kernels in a wet milling process, comprising
a) separating starch and/or gluten from fiber to provide a fiber fraction,
b) providing a fine fiber fraction by separating coarse fibers of the fiber fraction from fine fibers of the fiber fraction; and
c) contacting the fine fiber fraction with one or more hydrolytic enzymes.

2. The method according to claim 1, wherein the fine fiber fraction is provided by passing the fiber fraction through a filter, a screen or a sieve having a pore size of 1000 μm and removing fibers retained by the sieve or screen.

3. The method according to claim 1, wherein the fine fiber fraction is provided by
a) Passing the fiber fraction through a filter, a screen or a sieve having a pore size of 1000 μm and removing fibers retained by the filter, sieve or screen; and then
b) Passing the remaining part of the fiber fraction through a filter, a sieve or a screen having a pore size of 50 μm and collecting fibers retained by the filter, sieve or screen.

4. The method according to claim 1, wherein the fine fiber fraction is contacted with said one or more hydrolytic enzymes for a time period which is from 2-72 hours.

5. The method according to claim 1, wherein the fine fiber fraction is contacted with said one or more hydrolytic enzymes at a temperature which is within the range of 35-70° C.

6. The method according to claim 1, wherein the fine fiber fraction is contacted with said one or more hydrolytic enzymes when retained in an incubator or retention tank.

7. The method according to claim 6, wherein said the incubator or retention tank comprises one or more agitators configured to prevent settling of solids and/or fine fibers.

8. The method according to claim 6, wherein said incubator or retention tank is in fluent connection with a screen unit in said fiber wash procedure.

9. The method according to claim 1, comprising
a) feeding the fiber fraction to a fiber press (P) and subjecting it to pressing and filtration to provide pressed fiber and fiber press filtrate, and
b) feeding the fiber press filtrate to a space (V₁), and retaining the fiber press filtrate in said space (V₁) before optionally feeding it to said fiber washing system (F);

10. The method according to claim 9, wherein said space (V₁) has a volume which is in the range of 80 and 250 m³.

11. The method according to claim 1, wherein said one or more hydrolytic enzymes is/are selected from the group consisting of cellulases (EC 3.2.1.4), xylanases (EC 3.2.1.8) arabinofuranosidases (EC 3.2.1.55 (Non-reducing end alpha-L-arabinofuranosidases); EC 3.2.1.185 (Non-reducing end beta-L-arabinofuranosidases) cellobiohydrolase I (EC 3.2.1.150), cellobiohydrolase II (E.C. 3.2.1.91), cellobiosidase (E.C. 3.2.1.176), beta-glucosidase (E.C. 3.2.1.21), beta-xylosidases (EC 3.2.1.37).

12. The method according to claim 1, wherein the one or more hydrolytic enzymes comprise a GH10 polypeptide with xylanase activity and/or a GH11 polypeptide with xylanase activity.

13. The method according to claim 1, wherein the one or more hydrolytic enzymes comprise a GH61 polypeptide with arabinofuranosidase activity and/or a GH62 polypeptide with arabinofuranosidase activity.

14. A corn kernel wet milling system comprising
i) a fiber washing system (F)
ii) a fiber press (P),
iii) means for dosing one or more hydrolytic enzymes; and
iv) a space (V₁);
wherein said fiber washing system (F), said fiber press (P) and said space (V₁) are interconnected to allow a flow of a fiber fraction from the fiber washing system to the press, a flow of fiber press filtrate or a fine fiber fraction from the press to the space (V₁);
wherein the said means for dosing one or more hydrolytic enzymes is configured for dosing said enzymes to the fiber press filtrate or the fine fiber fraction
and said space (V₁) has a volume of at least 100 m³/1000 tonne corn processed per day.

15. The corn kernel wet milling system according to claim 14, wherein
said fiber washing system (F), said fiber press (P) and said space (V₁) are interconnected to allow a flow of a fiber fraction from the fiber washing system to the press, a flow of fiber press filtrate or fine fiber fraction from the press to the space (V₁) and a flow of fiber press filtrate or fine fiber fraction from the space (V₁) to the fiber washing system;
and
said space (V₁) is arranged at a position, which is between the fiber press and the fiber washing system.


SEQUENCE LISTING (35 sequences)

SEQ ID NO: 1 - protein, 302 aa, Penicillium capsulatum
NCALPSTYSWTSTSALANPKPGWTAIKDFTNVVFNNRHVVYASTTDTSGNYGAMSFGVFSDWPGMASASQNALSFAAVAPTLFYFQPKSIWVLAYQWGSSTFTYRTSSDPTNAYGWSSEQALFSGKVTGSSTGAIDQTLIGDATHMYLFFAGDNGKIYRSSMPISNFPGNFGTVSEVVLSDTQNNLFEAVQVYTVKGQNQYLMIVEAIGSEGRYFRSFTASSLGGLWTAQAASETKPFAGKANSGATWTNDISHGDLVRSNPDQTMTIDPCNLQFLYQGRNPGASGNYNTLPWRPGVLTLNN

SEQ ID NO: 2 - protein, 303 aa, Penicillium aurantiogriseum
DCALPSTYTWTSTGALANPKSGWTAIKDFTNVVVNNKHLVYASTTDASGNYGAMNFGPFSDWSGMATASQIKTSFNAVAPTLFYFQPKDIWVIAYQWGSSTFTYRTSSDPTNANGWSSEQALFSGKITAPDAAIDQTVIGDSTHMYLFFAGDNGKIYRSSMSIDKFPGNFGTSSEIVLSGARNDLFEAVQVYTVKGQNKYLMLVEAIGAQGQRYFRSFVSSSLGGKWEPQAASESKPFAGKANVGATWTKDFSHGDLVRTNPDQTMTVDPCNLQLLYQGRDPTATSSNYNTIPWQPAVLTLKK

SEQ ID NO: 3 - protein, 382 aa, Aspergillus clavatus
QQTLYGQCGGNGWTGPTQCVSGACCQIQNPWYSQCLPGSCSPSTTLTRVTTTATSTASTATSGTGGSLPSSFKWSSSGPLVDPKNDGRGIAALKDPSIVEVDGTYHVFASTATSAGYNMVYFNFTDFNQANNAPFFYLDKSPIGSGYRAAPQVFFFKPQNLWYLVYQNGNAAYSTNKDISNPAGWSAPKTFYSSQPSIITENIGNGYWVDMWVICDSANCHLFSSDDNGHLYRSQTTLANFPNGMTNTVIAMQDSNPNNLFEASNVYHVGGGKYLLIVEAIGSGGDRYFRSWTSTSLTGTWTALAASESNPFAGAKNVAFSGNVWTKSISHGEMIRDQVDQTLTISPCKLRYLYQGVDPAATGNYNSLPWKLALLTQTNSAC

SEQ ID NO: 4 - protein, 378 aa, Aspergillus clavatus
QQPLYAQCGGNGWTGSTQCVAGACCSSINAWYYQCLSGNCMPSTTMTTTATRTTSTSTSGPTGSLPPSFKWSSTNALVGPKNDGRNLAGIKDPSIIEVDGTYHVFASTAQASGYNLVYFNFTDFNQAGNAPFFYLDQSGIGTGYRAAPQVFYFQPQQLWYLIFQNGNAAYSTNKDISNPAGWSAPKNFFSSVPSIITQNIGNGYWVDMWVICDSSNCYLFSSDDNGHLYRSQTTLSNFPNGMGNTVIALSDSNPNNLFEASNVYRVGNEYLLIVEAIGSDGNRYFRSWTAPSLTGTWTGLANTEANPFARWNNVVFSGTAWTKSISHGEMVRSQVDQTMTISPCKLRYLYQGLSPTATGDYNSLPWKLALLTQTNSAC

SEQ ID NO: 5 - protein, 311 aa, Ustilago maydis
NPETERRRSCALPTTYRWTSSAPLAQPKDGWVSLKDFTHVPYNGQHLVYASYHDSTKYGSMAFSPFKHWADMATATQTGMTQAAVAPTVFYFTPKKLWFLVSQWGSAPFTYRTSTDPTKVNGWSAPQPLFTGKVADSGTGPIDQTVIADDRKVYLFFVADNGKVYRTSMAIGDFPANFGTASEVILSDTQAKLFEAVQVYTVAGQNQYLMIVEAQGTNGRYFRSFTANSLDGEWKVQAGSESAPFAGKANSGASWTNDVSHGDLIRSNPDQTMTIDPCRLQLLYQGRDKNKVPSSYDLAPYRPGLLTLYGL

SEQ ID NO: 6 - protein, 302 aa, Penicillium oxalicum
PVPSQGQYRWSSTGALAQPQHGWTSIKDFTNVVYNGKHLVYASVADSKGNYHSMNFGLFSDWSQMASASQNPMNFNAVAPTLFFFAPKNVWVLAYQWGANAFSYRTSNDPANANGWSSEHPLFTGKIANSGTGPIDQTLIGDNQNMYLFFAGDNGKIYRSSMPLNNFPGSFGGASEVILSDTTANLFEAVQVYKVAGENKYLMIVEAMGAHGRYFRSFTATSLNGKWTLNAGSEGAPFAGKANSGAGWTNDISHGDLVRTNPDQTMTVDMCNLQFLYQGRDPNANPTYNALPYRPGVLTLKH

SEQ ID NO: 7 - protein, 309 aa, Talaromyces pinophilus
SPVPEKRSGCALPSTYKWTSTGPLASPKSGLVALRDYSHVIYNGQHLVYGSTANTAGSYGSMNFGLFSDWSEMSSASQNTMSTGAVAPTIFYFAPKSVWILAYQWGPYAFSYRTSTDPSNANGWSSPQPLFTGTISGSSTGVIDQTVIGDSENMYLFFAGDNGHIYRASMPIGDFPGSFGSASTIVLSDSTNNLFEAVEVYTVEGQNQYLMIVEAIGANGRYFRSFTASSLGGTWTAQASTESNPFAGKANSGATWTNDISSGDLVRTNPDQTQTIDACNLQFLYQGRSTSSGGDYNLLPYQPGLLTLA

SEQ ID NO: 8 - protein, 438 aa, Streptomyces nitrosporeus
AASGALRGAGSGRCVDVTGGERTDGTTLQLYDCWGGTNQQWTSTDSGQLTVYGDKCLDVPGHATTPGTRVQIWGCSGGANQQWRVNSDGTVVGVESGLCLEAAGAGTANGTAVQLWTCNGGSNQKWTGLPATPPTDGTCSLPSAYRWTSTGVLAQPANGWAAVKDFTTVTHNGKHLVYASNVSGSSYGSMMFSPFTDWPDMASAGQTGMSQAAVAPTLFYFAPKNIWVLAYQWGAWPFIYRTSSNPADPNGWSSPQPLFTGSISGSDTGPIDQTLIADGQNMYLFFAGDNGKIYRASMPIGNFPGSFGSSYTTVMSDTKANLFEGVQVYKVKDRSQYLMIVEAMGANGRYFRSFTASSLNGTWTPQAATESSPFAGKANSGATWTNDISHGDLVRDNPDQTMTVDPCNLRFLYQGKAPDAGGEYNRLPWRPGVLTLRR

SEQ ID NO: 9 - protein, 446 aa, Artificial Sequence
  Mature sequence with His-tag
HHHHHHPRAASGALRGAGSGRCVDVTGGERTDGTTLQLYDCWGGTNQQWTSTDSGQLTVYGDKCLDVPGHATTPGTRVQIWGCSGGANQQWRVNSDGTVVGVESGLCLEAAGAGTANGTAVQLWTCNGGSNQKWTGLPATPPTDGTCSLPSAYRWTSTGVLAQPANGWAAVKDFTTVTHNGKHLVYASNVSGSSYGSMMFSPFTDWPDMASAGQTGMSQAAVAPTLFYFAPKNIWVLAYQWGAWPFIYRTSSNPADPNGWSSPQPLFTGSISGSDTGPIDQTLIADGQNMYLFFAGDNGKIYRASMPIGNFPGSFGSSYTTVMSDTKANLFEGVQVYKVKDRSQYLMIVEAMGANGRYFRSFTASSLNGTWTPQAATESSPFAGKANSGATWTNDISHGDLVRDNPDQTMTVDPCNLRFLYQGKAPDAGGEYNRLPWRPGVLTLRR

SEQ ID NO: 10 - protein, 438 aa, Artificial Sequence
  Mature sequence with His-tag
AAGGALRQAASGRCLDVPGAVQTDGTSVQIYDCWSGTNQQWTSTDANQLTVYGNKCLDVPGHATTAGTRVQIWSCSGGANQQWRVNSDGTVTGVESGLCLEAAGAATANGTAVQLGTCNQGSNQKWSGLTGTPPTDGSCSLPSTYRWSSTGVLAQPANGWAAVKDFTTVTYNGKHLVYASNVSGSSYGSMMFSPFTNWSDMASAGQSGMSQAAVAPTLFYFAPKNIWVLAYQWGASPFVYRTSSDPTNPNGWSSPQPLFTGSISGSDTGPIDQTLIADGQNMYLFFAGDNGKIYRASMPIGNFPGNFGSSYTTVMSDTKANLFEGVQVYKVQGQNQYLMIVEAMGANGRYFRSFTASSLNGSWAPQAATESNPFAGKANSGATWTNDISHGDLVRGNPDQTMTIDPCNLQLLYQGKSPTAGGPYDQLPWRPGVLSLQR

SEQ ID NO: 11 - protein, 446 aa, Artificial Sequence
  Mature sequence with His-tag
HHHHHHPRAAGGALRQAASGRCLDVPGAVQTDGTSVQIYDCWSGTNQQWTSTDANQLTVYGNKCLDVPGHATTAGTRVQIWSCSGGANQQWRVNSDGTVTGVESGLCLEAAGAATANGTAVQLGTCNQGSNQKWSGLTGTPPTDGSCSLPSTYRWSSTGVLAQPANGWAAVKDFTTVTYNGKHLVYASNVSGSSYGSMMFSPFTNWSDMASAGQSGMSQAAVAPTLFYFAPKNIWVLAYQWGASPFVYRTSSDPTNPNGWSSPQPLFTGSISGSDTGPIDQTLIADGQNMYLFFAGDNGKIYRASMPIGNFPGNFGSSYTTVMSDTKANLFEGVQVYKVQGQNQYLMIVEAMGANGRYFRSFTASSLNGSWAPQAATESNPFAGKANSGATWTNDISHGDLVRGNPDQTMTIDPCNLQLLYQGKSPTAGGPYDQLPWRPGVLSLQR

SEQ ID NO: 12 - protein, 318 aa, Aspergillus clavatus
ASLPRSFKWSSSAALVGPKNDGRHIEGIKDPSIVEVDGTYHVFASTAQASGYNLVYLSFTDFNKAHLAPFHYLDQTRIGKGYRAAPQVFYFKPHKLWYLVYQNGNAAYSTNPDISNPAGWTSPQNFFSGTPSIITHNMGRGAWVDMWTICDTRNCYLFSSDDNGHLYRSQTSLADFPHGMGNTAIALADRNKFSLFEASNVYHTGDGSYLLIVEAIGNDGQRYFRSWTASSLAGQWKPLADTESNPFARSNNVAFANGHAWTKSISHGEMIRTQTDQTMTISPCKLRYLYQGVDPAAKGDYNALPWKLGLLTQTNSAC

SEQ ID NO: 13 - protein, 326 aa, Artificial Sequence
  Mature sequence with His-tag
ASLPRSFKWSSSAALVGPKNDGRHIEGIKDPSIVEVDGTYHVFASTAQASGYNLVYLSFTDFNKAHLAPFHYLDQTRIGKGYRAAPQVFYFKPHKLWYLVYQNGNAAYSTNPDISNPAGWTSPQNFFSGTPSIITHNMGRGAWVDMWTICDTRNCYLFSSDDNGHLYRSQTSLADFPHGMGNTAIALADRNKFSLFEASNVYHTGDGSYLLIVEAIGNDGQRYFRSWTASSLAGQWKPLADTESNPFARSNNVAFANGHAWTKSISHGEMIRTQTDQTMTISPCKLRYLYQGVDPAAKGDYNALPWKLGLLTQTNSACRHHHHHHP

SEQ ID NO: 14 - protein, 302 aa, Aspergillus wentii
DCALPSTYSWTSTGSLADPKSGWTALKDFTNVVSNNKHIVYASTTDASGNYGSMNFASFSDWSDMASASQAATSFTAVAPTLLYFQPKSIWVLAYQWGSSTFTYRTSSDPTNANGWSSEKALFSGKITGSDTGAIDQTLIGDATNMYLFFAGDNGKIYRSSMPIANFPGDFGTASEVVLSDSRNNLFEAVQVYTVEGQNQYLMIVEAIGTNGRYFRSFTASSLDGSWTEQAASENNPFAGKANSGATWTNDISHGDLVRNNPDQTMTIDPCNLQFLYQGRDASAGGNYNTLPWRPGVLTLKH

SEQ ID NO: 15 - protein, 311 aa, Artificial Sequence
  Mature sequence with His-tag
DCALPSTYSWTSTGSLADPKSGWTALKDFTNVVSNNKHIVYASTTDASGNYGSMNFASFSDWSDMASASQAATSFTAVAPTLLYFQPKSIWVLAYQWGSSTFTYRTSSDPTNANGWSSEKALFSGKITGSDTGAIDQTLIGDATNMYLFFAGDNGKIYRSSMPIANFPGDFGTASEVVLSDSRNNLFEAVQVYTVEGQNQYLMIVEAIGTNGRYFRSFTASSLDGSWTEQAASENNPFAGKANSGATWTNDISHGDLVRNNPDQTMTIDPCNLQFLYQGRDASAGGNYNTLPWRPGVLTLKHTRAHHHHHH

SEQ ID NO: 16 - protein, 364 aa, Acrophialophora fusispora
ACSLPSSYRWASTGPLANPKSGWYSLKDFTHVPYNGKHLVYASNYAGSAYGSMNFGLFSNWSDMASASQNSMNAAAVAPTLFYFAPKNIWVLASQWGATPFFYRTSTDPTNPNSWSSNQPLFTGSISDSSTGPIDQTLIGDANYMYLFFAGDNGKIYRSRMPIGNFPGSFGSSYEVILSGSRNDFFEAVQVYTVTGQSSPLYLMIIESIGSRGRYFRSYTATNLGGSWSPQATSESSPFAGAANSGATWTNDISHGDLIRSGPDQTMPIDPCNLQLLYQGLVGTNSDYNKLPYRPGLLTLQNPVGGGGTPTTTTSKPPATTTSTGGGGTAPQYAQCGGQGYTGPTVCASPYKCTYSNPWYSQCL

SEQ ID NO: 17 - protein, 373 aa, Artificial Sequence
  Mature sequence with His-tag
ACSLPSSYRWASTGPLANPKSGWYSLKDFTHVPYNGKHLVYASNYAGSAYGSMNFGLFSNWSDMASASQNSMNAAAVAPTLFYFAPKNIWVLASQWGATPFFYRTSTDPTNPNSWSSNQPLFTGSISDSSTGPIDQTLIGDANYMYLFFAGDNGKIYRSRMPIGNFPGSFGSSYEVILSGSRNDFFEAVQVYTVTGQSSPLYLMIIESIGSRGRYFRSYTATNLGGSWSPQATSESSPFAGAANSGATWTNDISHGDLIRSGPDQTMPIDPCNLQLLYQGLVGTNSDYNKLPYRPGLLTLQNPVGGGGTPTTTTSKPPATTTSTGGGGTAPQYAQCGGQGYTGPTVCASPYKCTYSNPWYSQCLTRAHHHHHH

SEQ ID NO: 18 - protein, 436 aa, Streptosporangium sp-60756 60
AGAAAGCRVDYTVSNQWPGGFGANVNITNLGDPINGWRLTWSFPAGQTITQLWSGSHTQSGSQVTVTNVDYNAGLPTGGSANFGFNGSFNGSNPAPTSFALNGVTCTGGVTASPSPSTSPSTGPSPSSTPTSPGTCALPSTYRWTSTGPLANPKSGWVSLKDFTNVVHNGKHLVYATTHDTGTSWGSMNFSPFTNWSDMASAGQNKMNFSTVAPTLFYFAPKNIWVLAYQWGGTAFSYRTSSDPTNANGWSAQQTLFTGSISGSGTGPIDQTLIGDGTNMYLFFAGDNGKIYRASMPIGNFPGSFGSNYTTIMSDTTNNLFEGVEVYKLQGQNKYLMLVEAIGSQGRYFRSFTATSLDGTWTPQAATEGNPFAGKANSGATWTNDISHGDLVRSNPDQTKTVDPCNLQLLYQGRSPNSGGDYGLLPYRPGVLTLQR

SEQ ID NO: 19 - protein, 444 aa, Artificial Sequence
  Mature sequence with His-tag
HHHHHHPRAGAAAGCRVDYTVSNQWPGGFGANVNITNLGDPINGWRLTWSFPAGQTITQLWSGSHTQSGSQVTVTNVDYNAGLPTGGSANFGFNGSFNGSNPAPTSFALNGVTCTGGVTASPSPSTSPSTGPSPSSTPTSPGTCALPSTYRWTSTGPLANPKSGWVSLKDFTNVVHNGKHLVYATTHDTGTSWGSMNFSPFTNWSDMASAGQNKMNFSTVAPTLFYFAPKNIWVLAYQWGGTAFSYRTSSDPTNANGWSAQQTLFTGSISGSGTGPIDQTLIGDGTNMYLFFAGDNGKIYRASMPIGNFPGSFGSNYTTIMSDTTNNLFEGVEVYKLQGQNKYLMLVEAIGSQGRYFRSFTATSLDGTWTPQAATEGNPFAGKANSGATWTNDISHGDLVRSNPDQTKTVDPCNLQLLYQGRSPNSGGDYGLLPYRPGVLTLQR

SEQ ID NO: 20 - protein, 302 aa, Acrophialophora fusispora
QCPLPSTYRWKSTGVLANPKSGWVSLKDFTVAPYNGKHLVYATTHDTGSSWGSMNFGLFSSWSDMATAPQNGMNQGTVAPTLFYFKPKDIWVLAYQWGPTTFSYKTSKDPTNANGWGSAQTLFSGKISGSSTGAIDQTVIGDDTNMYLFFAGDNGKIYRASMPIDRFPGSFGDQYQTILSDSTNNLFEAVQVYKLQGLNKYLMIVEAIGSNGRYFRSFTADRLDGQWTPQAATESNPFAGKANSGATWTNDISHGELIRVSADQTFTVDPCNLQLLYQGRSPSSGGDYGKLPYRPGLLTLQR

SEQ ID NO: 21 - protein, 311 aa, Artificial Sequence
  Mature sequence with His-tag
QCPLPSTYRWKSTGVLANPKSGWVSLKDFTVAPYNGKHLVYATTHDTGSSWGSMNFGLFSSWSDMATAPQNGMNQGTVAPTLFYFKPKDIWVLAYQWGPTTFSYKTSKDPTNANGWGSAQTLFSGKISGSSTGAIDQTVIGDDTNMYLFFAGDNGKIYRASMPIDRFPGSFGDQYQTILSDSTNNLFEAVQVYKLQGLNKYLMIVEAIGSNGRYFRSFTADRLDGQWTPQAATESNPFAGKANSGATWTNDISHGELIRVSADQTFTVDPCNLQLLYQGRSPSSGGDYGKLPYRPGLLTLQRTRAHHHHHH

SEQ ID NO: 22 - protein, 405 aa, Talaromyces leycettanus
MVHLSSLALALAAGSQLAQAAGLNTAAKAIGKLYFGTATDNPELSDSTYMQETDNTDDFGQLTPANSMKWDATEPSQNTFTFTNGDQIANLAKSNGQMLRCHNLVWYNQLPSWVTSGSWTNATLLAAMKNHITNVVTHYKGQCYAWDVVNEALNDDGTYRSNVFYQYIGEAYIPIAFATAAAADPNAKLYYNDYNIEYPGAKATAAQNIVKMVKAYGAKIDGVGLQSHFIVGSTPSQSSQQSNMAAFTALGVEVAITELDIRMTLPSTSALLAQQSTDYQSTVSACVNTPKCIGITLWDWTDKYSWVPNTFSGQGDACPWDSNYQKKPAYYGILTALGGSASTSTTTTLVTSTRTSTTTSTSATSTSTGVAQHWGQCGGIGWTGPTTCASPYTCQELNPYYYQCL

SEQ ID NO: 23 - protein, 398 aa, Trichophaea saccata
MRTFSSLLGVALLLGAANAQVAVWGQCGGIGYSGSTTCAAGTTCVKLNDYYSQCQPGGTTLTTTTKPATTTTTTTATSPSSSPGLNALAQKSGRYFGSATDNPELSDAAYIAILSNKNEFGIITPGNSMKWDATEPSRGSFSFTGGQQIVDFAQGNGQAIRGHTLVWYSQLPSWVTSGNFDKATLTSIMQNHITTLVSHWKGQLAYWDVVNEAFNDDGTFRQNVFYTTIGEDYIQLAFEAARAADPTAKLCINDYNIEGTGAKSTAMYNLVSKLKSAGVPIDCIGVQGHLIVGEVPTTIQANLAQFASLGVDVAITELDIRMTLPSTTALLQQQAKDYVSVVTACMNVPRCIGITIWDYTDKYSWVPQTFSGQGDACPWDANLQKKPAYSAIASALAA

SEQ ID NO: 24 - protein, 384 aa, Aspergillus aculeatus
VGLDQAAVAKGLQYFGTATDNPELTDIPYVTQLNNTADFGQITPGNSMKWDATEPSQGTFTFTKGDVIADLAEGNGQYLRCHTLVWYNQLPSWVTSGTWTNATLTAALKNHITNVVSHYKGKCLHWDVVNEALNDDGTYRTNIFYTTIGEAYIPIAFAAAAAADPDAKLFYNDYNLEYGGAKAASARAIVQLVKNAGAKIDGVGLQAHFSVGTVPSTSSLVSVLQSFTALGVEVAYTEADVRILLPTTATTLAQQSSDFQALVQSCVQTTGCVGFTIWDWTDKYSWVPSTFSGYGAALPWDENLVKKPAYNGLLAGMGVTVTTTTTTTTATATGKTTTTTTGATSTGTTAAHWGQCGGLNWSGPTACATGYTCTYVNDYYSQCL

SEQ ID NO: 25 - protein, 288 aa, Clostridium acetobutylicum
AMSHSKFVGNIIAGSIPSNFDTYWNQVTPENATKWGAIEYGRGNYNWGSADLIYNYARSKNMPFKFHNLVWGSQQLTWLSNLSPQDQKSEVSKWIAAAGQRYSGSAFVDVVNEPLHTQPSYKNALGGDGSTGYDWIVWSYQQARKAFPNSKLLINEYGIIGDPNAAANYVKIINVLKSKGLIDGIGIQCHYFNMDNVSVGTMNYVLNMLSNTGLPIYVSELDMTGDDSTQLARYQQKFPVLYQNPNVKGITLWGYMQGQTWNSGTYLVNSNGTERPALKWLRSYLASH

SEQ ID NO: 26 - protein, 308 aa, Aspergillus aculeatus
NPIEPRQASVSIDAKFKAHGKKYLGTIGDQYTLNKNAKTPAIIKADFGQLTPENSMKWDATEPNRGQFSFSGSDYLVNFAQSNGKLIRGHTLVWHSQLPSWVQSISDKNTLIQVMQNHITTVMQRYKGKVYAWDVVNEIFNEDGSLCQSHFYNVIGEDYVRIAFETARAVDPNAKLYINDYNLDSASYPKLTGLVNHVKKWVAAGVPIDGIGSQTHLSAGAGAAVSGALNALAGAGTKEVAITELDIAGASSTDYVNVVKACLNQPKCVGITVWGSSDPDSWRSSSSPLLFDSNYNPKAAYTAIANAL

SEQ ID NO: 27 - protein, 195 aa, Thermomyces lanuginosus
RQTTPNSEGWHDGYYYSWWSDGGAQATYTNLEGGTYEISWGDGGNLVGGKGWNPGLNARAIHFEGVYQPNGNSYLAVYGWTRNPLVEYYIVENFGTYDPSSGATDLGTVECDGSIYRLGKTTRVNAPSIDGTQTFDQYWSVRQDKRTSGTVQTGCHFDAWARAGLNVNGDHYYQIVATEGYFSSGYARITVADVG

SEQ ID NO: 28 - protein, 203 aa, Dictyoglomus thermophilum
QTSITLTSNASGTFDGYYYELWKDTGNTTMTVYTQGRFSCQWSNINNALFRTGKKYNQNWQSLGTIRITYSATYNPNGNSYLCIYGWSTNPLVEFYIVESWGNWRPPGATSLGQVTIDGGTYDIYRTTRVNQPSIVGTATFDQYWSVRTSKRTSGTVTVTDHFRAWANRGLNLGTIDQITLCVEGYQSSGSANITQNTFSQGS

SEQ ID NO: 29 - protein, 182 aa, Paenibacillus Pabuli
TDYWQNWTDGGGTVNAVNGSGGNYSVNWQNTGNFVVGKGWTYGTPNRVVNYNAGVFSPSGNGYLTFYGWTRNALIEYYVVDNWGTYRPTGTYKGTVTSDGGTYDIYTTMRYNQPSIDGYSTFPQYWSVRQSKRPIGVNSQITFQNHVNAWASKGMYLGNSWSYQVMATEGYQSSGSSNVTVW

SEQ ID NO: 30 - protein, 183 aa, Geobacillus stearothermophilus
ATDYWQYWTDGGGMVNAVNGPGGNYSVTWQNTGNFVVGKGWTVGSPNRVINYNAGIWEPSGNGYLTLYGWTRNALIEYYVVDSWGTYRPTGNYKGTVNSDGGTYDIYTTMRYNAPSIDGTQTFQQFWSVRQSKRPTGSNVSITFSNHVNAWRSKGMNLGSSWAYQVLATEGYQSSGRSNVTVW

SEQ ID NO: 31 - protein, 183 aa, Geobacillus stearothermophilus; identical to SEQ ID NO: 30

SEQ ID NO: 32 - protein, 299 aa, Streptomyces beijiangensis
DTVVNSNQTGTNNGYYYSHWSDGGGSVSMTLGSGGNYGYQWSNVGNFVGGKGWSTGGRKSVNYSGSFNPSGNAYLALYGWTTNPLVEYYVVENFGTYRPTGTFKGTVTSDGGTYDIYETTRVNQPSIEGTKTFKQYWSVRQSKRTGGTITTGNHFDAWSSHGMSMGSFNYMIMATEGYQSSGSSNITVSEGSSGGGTGGGGTGGGTGGGGSGGCTATLSAGDKWSDRYNLNVSVSGAGNWTVTMKVPSPEKVLSTWNVSAAYPDSQTLVAKSNGSGSNWGATIQTNGSWTWPTVTCSAG

SEQ ID NO: 33 - protein, 299 aa, Streptomyces beijiangensis; identical to SEQ ID NO: 32

SEQ ID NO: 34 - protein, 188 aa, Fusarium oxysporum
TQPTTGTSGGYYFSFWTDTPNSVTYTNGNGGQFSMQWSGNGNHVGGKGWMPGTSRTIKYSGSYNPNGNSYLAVYGWTRNPLIEYYIVENFGTYNPSSGGQKKGEVNVDGSVYDIYVSTRVNAPSIDGNKTFQQYWSVRRNKRSSGSVNTGAHFQAWKNVGLNLGTHDYQILAVEGYYSSGSASMTVSQ

SEQ ID NO: 35 - protein, 189 aa, Aspergillus clavatus
AGTPSSTGWNNGYYYSFWTDNGGTVNYQNGNGGSYSVQWKDTGNFVGGKGWNPGSARTINYSGSFNPSGNAYLTVYGWTTNPLVEYYIVENYGTYNPGNGGTYRGSVYSDGANYNIYTATRYNAPSIEGDKTFTQYWSVRQSKRTGGTVTTANHFNAWAQLGMSLGTHNYQIVATEGYQSSGSSSITVY